(19) United States
(12) Patent Application Publication
(10) Pub. No.: US 2012/0070442 A1
(43) Pub. Date: Mar. 22, 2012

(54) METHODS AND COMPOSITIONS FOR TREATING CANCER

(75) Inventors: Glen Joel Weiss, Phoenix, AZ (US); Lynne Bemis, Golden, CO (US); Paul A. Bunn, JR., Steamboat, CO (US); Wilber A/ Franklin, Denver, CO (US)

(73) Assignee: The Regents of the University of Colorado, a body corporate, Denver, CO (US)

(21) Appl. No.: 12/672,557

(22) PCT Filed: Aug. 11, 2008
(86) PCT No.: PCT/US2008/072787

§ 371 Date: Oct. 17, 2011

Related U.S. Application Data
(60) Provisional application No. 60/954,981, filed on Aug. 9, 2007.

(51) Int. Cl. A61K 39/395; A61P 35/00; A61K 31/713; A61K 31/7088; C12Q 1/68; A61K 31/5377

(52) U.S. Cl. 424/155.1; 435/6.12; 435/6.11; 514/234.5; 514/44 A; 514/44 R; 424/174.1; 435/6.13

(57) ABSTRACT

Provided herein are methods for identifying a cancer patient responsive to treatment with an EGFR tyrosine kinase inhibitor. One method comprises obtaining a biopsy from the patient and measuring the number of copies of miR-128b in DNA extracted from the biopsy. A patient responsive to EGFR tyrosine kinase inhibitor treatment has a cancer with less than two copies of miR-128b DNA. Another method comprises measuring miR-128b or miR-128a level in a biopsy obtained from the patient and comparing that level to miR-128b or miR-128a level in a normal tissue sample. A patient responsive to treatment with an EGFR tyrosine kinase inhibitor has a cancer expressing a lower level of miR-128b or miR-128a relative to normal tissue. Further provided herein are methods for treating cancer in a patient in need thereof. One method comprises measuring the level of miR-128b or miR-128a in a biopsy obtained from the patient and administering to the patient an EGFR tyrosine kinase inhibitor. Another method comprises measuring the number of copies of miR-128b in DNA extracted from a biopsy obtained from the patient and administering to the patient an EGFR tyrosine kinase inhibitor. A further method comprises administering to a cancer patient an EGFR tyrosine kinase inhibitor and an miR-128b inhibitor, administering an miR-128a mimic, or administering an miR-128b mimic. Also provided herein are compositions used to treat cancer in a patient. The compositions comprise an EGFR tyrosine kinase inhibitor and an miR-128b inhibitor (or an miR-128a inhibitor), and the cancer is characterized as having 2 or more copies of miR-128b DNA at the cellular level.

### CROSS-REFERENCES TO RELATED APPLICATIONS

The application claims the benefit of U.S. Provisional Application No. 60/954,981, filed Aug. 9, 2007, the contents of which is herein incorporated by reference in its entirety for all purposes.

### STATEMENT AS TO RIGHTS TO INVENTIONS MADE UNDER FEDERALLY SPONSORED RESEARCH OR DEVELOPMENT

The present invention was developed with funds from the National Cancer Institute-P50-58187 Specialized Program of Research Excellence in Lung Cancer (SPORE). The U.S. government has certain rights to the invention.

### BACKGROUND OF THE INVENTION

Lung carcinoma remains the leading cause of cancer death worldwide for both men and women. Non-small cell lung cancer (NSCLC) accounts for approximately 86% of lung cancer cases and presents in advanced stage about 75% of the time (Weiss et al. 2006 Oncology 20: 1515). NSCLC includes squamous cell carcinoma, adenocarcinoma (including bronchioloalveolar carcinoma), and large cell carcinoma. Other less common types of NSCLC are pleomorphic, carcinoid tumor, salivary gland carcinoma, and unclassified carcinoma.

Epidermal growth factor receptor (EGFR) is a transmembrane receptor normally involved in cell proliferation. The receptor has an extracellular ligand binding domain, a transmembrane domain, and an intracellular domain with tyrosine kinase activity. Phosphorylation of the EGFR activates downstream signaling proteins involved in signal transduction cascades, including MAPK, Akt, and INK pathways, resulting in DNA synthesis and cell proliferation. The signaling pathways regulate cell migration, adhesion, and proliferation. Thus, overexpression of EGFR and/or its ligands in cancer cells facilitates cancer growth and metastasis and is an indicator of poor outcome.

Strategies have been developed to target and inhibit the EGFR family, including the use of monoclonal antibodies, which either bind the ligand or compete with the ligand for the extracellular domain of the receptor; inhibitors of receptor dimerization; small-molecule inhibitors of the intracellular tyrosine kinase domain (EGFR-TKI) including gefitinib and erlotinib; antisense oligonucleotides; and inhibitors of the EGFR downstream signaling network. By interfering with cell signaling pathways involved in cell proliferation, inhibition of EGFR tyrosine kinase represents a novel approach to the treatment of solid tumors. Gefitinib and erlotinib are small molecules that reversibly target EGFR tyrosine kinase, and each demonstrates effectiveness when used to treat patients with NSCLC. Gefitinib inhibits EGFR-TK by binding to the adenosine triphosphate (ATP)-binding site of the enzyme, preventing autophosphorylation of the EGFR homodimers. This inhibits the function of the EGFR-TK in activating the signaling cascade. Like gefitinib, erlotinib specifically targets the EGFR-TK and reversibly binds to the ATP binding site of the receptor.

However, it is difficult to predict a survival benefit of treatment with EGFR-TKIs; even using immuno-histochemistry to identify patients with cancers having relatively higher EGFR protein levels is insufficient (Parra et al. 2004 Brit. J. of Cancer 91: 208; Bailey et al. 2003 Proc. Am. Assoc. Cancer Res. 44: 170A). Whether EGFR mutation in the tyrosine kinase domain (EGFR exons 18-21), high EGFR gene copy number, or gene amplification by fluorescence in situ hybridization (FISH) correlate better with response and survival after EGFR-TKIs is unclear. These events are not mutually exclusive: up to 24% of patients have concurrent mutation and high copy number (Hirsch et al. 2006 J. Clin. Oncol. 24: 5034).

In western NSCLC patient populations, EGFR mutation prevalence is 10-23% compared to 22-45% high EGFR copy number and/or amplification in the Japanese population (Dziadzuiskzo et al. 2006 Clin. Cancer Res. 12: 4409s).

As such, there is a need in the art for improving treatment of cancer patients, and particularly for improving treatment of cancer patients with EGFR expressing tumors. Furthermore there is a need for better methods to predict those patients who will respond to EGFR targeted therapies.

### BRIEF SUMMARY OF THE INVENTION

The present invention provides methods and compositions for the identification and treatment of cancer, and in particular, EGFR expressing cancers.

Provided herein is a method for identifying a cancer patient responsive to treatment with an EGFR tyrosine kinase inhibitor. The method includes detecting a genomic loss of miR-128b or miR-128a in a cancer biopsy obtained from the patient. The genomic loss of miR-128b or miR128a indicates the cancer patient is responsive to treatment with an EGFR tyrosine kinase inhibitor.

There is still further provided a method for treating cancer in a patient in need thereof. In some embodiments, the method comprises measuring the level of miR-128b or miR-128a in a sample (e.g. a biopsy) having cancerous tissue obtained from the patient and administering to the patient an EGFR tyrosine kinase inhibitor. In other embodiments, the method comprises measuring the number of copies of miR-128b in DNA extracted from a sample (e.g. a biopsy) having cancerous tissue obtained from the patient and administering to the patient an EGFR tyrosine kinase inhibitor.

In still other embodiments, the method comprises administering to a cancer patient a composition comprising an EGFR tyrosine kinase inhibitor and a miR-128b inhibitor or miR-128a inhibitor. In further embodiments, the method comprises administering to a cancer patient a composition comprising a miR-128b mimic or a miR-128a mimic.

There is also provided compositions used to treat cancer in a patient. In one embodiment, the composition comprises an EGFR tyrosine kinase inhibitor and a miR-128b inhibitor. Such compositions are typically used to treat a patient having cancer characterized by having a ratio of miR-128b to CFTR copies of DNA>0.5 at the cellular level. Additional compositions include an EGFR tyrosine kinase inhibitor and a miR-128a inhibitor, or a combination of an EGFR tyrosine kinase inhibitor, a miR-128a inhibitor, and a miR-128b inhibitor.

Provided herein are methods for identifying cancer therapeutics. In one embodiment, the method comprises screening for compounds that target a miR-128b binding site or miR-128a binding site on a 3′ untranslated region of EGFR mRNA. In another embodiment, the method comprises screening for compounds that inhibit miR-128b, and the resultant therapeutic used in combination with an EGFR tyrosine kinase inhibitor to treat a cancer that expresses miR-128b.

Also provided herein are methods for identifying a patient or patient population predisposed to cancer. The method comprises measuring the level of miR-128b, number of miR-128b DNA copies, or both in a sample (e.g. a biopsy) obtained from the patient.

Further features and benefits of the invention will be apparent to one skilled in the art from reading this specification.

### BRIEF DESCRIPTION OF THE DRAWINGS

FIG. 1 shows the potential miR-128b binding sites on the EGFR-3′ untranslated region (SEQ ID NO: 17).

FIG. 2 represents a Western blot analysis of EGFR, p-EGFR, and p-AKT from the H157 cell line normalized to actin.

FIG. 3 illustrates EGFR, p-EGFR, and p-AKT expression in five cell lines treated with miR-128b inhibitor or miR-128b mimic relative to expression in the respective untreated control.

FIG. 4 illustrates Western blot analysis of GFP expression data in H157 cells transfected with GFP constructs compared to cells transfected with GFP-EGFR 3′untranslated region constructs.

FIG. 5 illustrates relative amounts of GFP protein, mRNA, and DNA copy in cell lines transfected with GFP constructs or GFP-EGFR 3′untranslated region constructs.

FIG. 6 illustrates overall survival of patients having cancer exhibiting miR-128b deletion relative to patients having cancer with normal or amplified miR-128b.

### DETAILED DESCRIPTION OF THE INVENTION

This detailed description is intended only to acquaint others skilled in the art with Applicants' invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent, and may be variously modified.

### Definitions

The following definitions are provided to facilitate understanding of certain terms used frequently herein and are not meant to limit the scope of the present disclosure.

The phrase “amino acid” as used herein refers to any of the twenty naturally occurring amino acids as well as any modified amino acids. Modifications can include natural processes such as posttranslational processing, or chemical modifications which are known in the art. Modifications include, but are not limited to, phosphorylation, ubiquitination, acetylation, amidation, glycosylation, covalent attachment of flavin, ADP-ribosylation, cross linking, iodination, methylation, and the like.

The word “antibody” as used herein refers to a Y-shaped molecule having a pair of antigen binding sites, a hinge region, and a constant region, as well as fragments thereof (i.e. antibody fragments). For example, the term antibody includes antigen binding fragments (Fab), chimeric antibodies, antibodies having a human constant region coupled to a murine antigen binding region, and fragments thereof, as well as other well known recombinant antibodies are contemplated herein.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (i.e., prostate, lymph node, liver, bone marrow, blood cell), the size and type of the tumor (i.e., solid or suspended (i.e., blood or ascites)), among other factors. Representative biopsy techniques include excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. Biopsy techniques are discussed, for example, in Harrison's Principles of Internal Medicine, Kasper, et al., eds., 16th ed., 2005, Chapter 70, and throughout Part V. The tissue is then available for diagnostic or chemical analysis. A biopsy can contain cancerous cells/tissue or normal cells/tissue. A “cancer biopsy” is a biopsy containing cancerous cells.

The words “complementary” or “complementarity” refers to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The terms “identical” or percent “identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., below.

The word “expression” as used herein refers to transcription and translation occurring within a cell. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

The phrase “genetically engineered” refers to any recombinant DNA or RNA method used to create a eukaryotic cell that expresses a target protein at elevated levels, at lowered levels, or in a mutated form. In other words, the cell has been transfected, transformed, or transduced with a recombinant polynucleotide, and thereby altered so as to cause the cell to alter expression of the desired proteins. Methods and vectors for genetically engineering host cells are well known in the art; for example, various techniques are illustrated in Current Protocols in Molecular Biology, Ausubel, et al., eds. (Wiley & Sons, New York, N.Y., 1988 and quarterly updates). Genetic engineering techniques include, but are not limited to, expression vectors, targeted homologous recombination and gene activation (see, for example, U.S. Pat. No. 5,272,071 to Chappel) and trans activation by engineered transcription factors (see, for example, Segal et al., 1999, Proc. Natl. Acad. Sci. USA 96(6): 2758-2763).

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

“Antisense,” “siRNA,” or “RNAi” refers to a nucleic acid that forms a double stranded RNA, which double stranded RNA has the ability to reduce or inhibit expression of a gene or target gene when expressed in the same cell as the gene or target gene. The complementary portions of the nucleic acid that hybridize to form the double stranded molecule typically have substantial or complete identity. In one embodiment, an antisense nucleic acid, siRNA or RNAi refers to a nucleic acid that has substantial or complete identity to a target gene and forms a double stranded siRNA. Typically, the nucleic is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

The word “polynucleotide” refers to a linear sequence of nucleotides. The nucleotides can be ribonucleotides, deoxyribonucleotides, or a mixture of both. Examples of polynucleotides contemplated herein include single and double stranded DNA, single and double stranded RNA (including miRNA), and hybrid molecules having mixtures of single and double stranded DNA and RNA. The polynucleotides described herein may contain one or more modified nucleotides.

The words “protein”, “peptide”, and “polypeptide” are used interchangeably to denote an amino acid polymer or a set of two or more interacting or bound amino acid polymers.

The term “treating” means ameliorating, suppressing, eradicating, and/or delaying the onset of the disease being treated.

### EGFR Expressing Cancer Cells

The surface of most normal cells typically expresses EGFR, however, mutations in the EGFR binding domain or mutations at the regulatory level can result in increased levels of EGFR and/or activated EGFR. Binding of a ligand to the receptor induces dimerization of the receptor with another EGFR or EGFR family member. Dimerization results in autophosphorylation of five tyrosine residues in the tyrosine kinase domain, and leads to activation of signaling pathways responsible for promoting cell growth, DNA synthesis, and the expression of oncogenes. Amplified EGFR signaling induces uncontrolled cell growth and malignancy.

### MiR-128a and b

MicroRNAs (miRNA) are single-stranded RNA molecules of about 21-23 nucleotides in length and are involved in crucial biologic processes such as proliferation, differentiation, development, and apoptosis (Calin and Croce, 2006, Nature Rev. Cancer 6: 857). miRNAs are encoded by genes transcribed from DNA but not translated into protein (non-coding RNA) and are instead processed from primary transcripts known as pri-miRNA to short stem-loop structures called pre-miRNA and finally to functional miRNA. Mature miRNA molecules are partially complementary to one or more messenger RNA (mRNA) molecules, typically at a site in the 3′ UTR of the mRNA. Annealing of the miRNA to mRNA inhibits translation, effectively downregulating gene expression. In some cases, however, annealing of the miRNA to mRNA facilitates cleavage of the mRNA by triggering the degradation of the mRNA transcript through a process similar to RNA interference (RNAi). In other cases, the miRNA complex blocks protein translation machinery or otherwise prevents protein translation without causing the mRNA to be degraded. miRNAs can also target methylation of genomic sites which correspond to targeted mRNAs.

As disclosed herein and in the Examples below, the inventors identified miRNA-128a and miRNA-128b as miRNAs involved in EGFR regulation miR-128a (hsa-mir-128a MI0000447) is found on chromosome 2 while miR128b (hsa-mir-128b MI0000727) is found on chromosome 3p. The DNA and RNA sequences of the miRNAs as well as the RNA sequences for the mature miRNAs are shown in Table 5. miR-128a and miR-128b differ at the mature miRNA by one base at the 3′ end. Other differences appear in the pre-mRNA sequences outside the sequence encompassing the mature miRNA.

### Methods of Ascertaining Responsiveness to Treatment

It is demonstrated herein that deletion of miR-128b copies at the genomic level unexpectedly correlates with clinical response and survival with EGFR tyrosine kinase inhibitor (e.g. gefitinib) treatment.

Provided herein is a method for identifying a cancer patient responsive to treatment with an EGFR tyrosine kinase inhibitor. The method includes detecting a genomic loss of miR-128b or miR-128a in a cancer biopsy obtained from the patient. The genomic loss of miR-128b or miR128a indicates the cancer patient is responsive to treatment with an EGFR tyrosine kinase inhibitor. In some embodiments, the method includes detecting a genomic loss of miR-128b in a cancer biopsy obtained from the patient. The genomic loss of miR-128b indicates the cancer patient is responsive to treatment with an EGFR tyrosine kinase inhibitor. By “responsive to treatment with an EGFR tyrosine kinase inhibitor” is meant that administration of an EGFR tyrosine kinase inhibitor would not result in remission of the cancer.

Also provided is determining whether a cancer patient is responsive to treatment with an EGFR tyrosine kinase inhibitor. The method includes determining whether a genomic loss of miR-128b or miR-128a is present in a cancer biopsy obtained from the patient. The presence of a genomic loss indicates the cancer patient is responsive to treatment with an EGFR tyrosine kinase inhibitor. The absence of a genomic loss indicates the cancer patient is not responsive to treatment with an EGFR tyrosine kinase inhibitor.

The term “genomic loss,” as used herein, means the loss of normal function of a gene due to changes at the chromosomal level. Genomic loss includes loss of heterozygosity (“LOH”), which refers to the absence of heterozygosity at a locus (e.g. the miR-128b locus at chromosome 3p) in a cancer cell. Thus, detecting a genomic loss may include determining whether a cancer patient (e.g. a lung cancer patient) possesses a loss of heterozygosity (“LOH”) of miR-128b, wherein a cancer patient having LOH of miR-128b is indicative of a cancer patient that is responsive to treatment with an EGFR inhibitor. In some embodiments, the determining of whether a cancer patient possesses a loss of heterozygosity (“LOH”) of miR-128b includes measuring the number of copies of miR-128b DNA within a sample (e.g. a biopsy or a cancer biopsy) obtained from the cancer patient. The number of copies of miR-128b DNA may be determined by measuring the ratio of miR-128b DNA to an unaffected gene (i.e. a gene whose DNA copy number is not affected by the lung cancer disease state) such as CFTR, beta-actin, or tubulin DNA copy. Where the number of copies of miR-128b DNA is determined by measuring the ratio of miR-128b DNA to an unaffected gene, the number of copies of miR-128b DNA may be referred to as the relative number of copies of miR-128b DNA. For example, a ratio of <0.5 of miR-128b DNA to an unaffected gene indicates the cells within the sample have less than two copies of miR-128b DNA per cell thereby determining that the lung cancer patient has LOH of miR-128b and is responsive to treatment with an EGFR inhibitor. Any applicable method may be used to determine the relative number of copies of miR-128b DNA within a sample, such as quantitative PCR and other such methods described herein.

Thus, in some embodiments, the method of identifying a cancer patient responsive to treatment with an EGFR inhibitor includes directly measuring miR-128b levels, miR-128a levels, and/or miR-128b DNA copies in a biopsy obtained from the patient. The biopsy according to this embodiment contains cancerous cells and/or tissue. As disclosed herein, miR-128b regulates the level of expression of EGFR in cancer cells. While not wishing to be bound by theory, it is believed that a cancer expressing miR-128b or miR-128a will not respond as well to treatment with EGFR tyrosine kinase inhibitors as the levels of EGFR protein are suppressed by the microRNAs. Thus, the method may include measuring miR-128b or miR-128a levels in a biopsy obtained from the patient.

In other embodiments, the method concludes obtaining a biopsy (e.g. cancer biopsy) from the patient, and measuring the number of copies of miR-128a or miR-128b in DNA extracted from the biopsy. A patient considered responsive to treatment may have a deletion of miR-128b per cancer cell (also referred to herein as a loss of heterozygosity (i.e. LOH)). This can be determined by measuring the ratio of miR-128b to an unaffected gene such as CFTR, beta-actin, or tubulin DNA copy. A ratio of <0.5 indicates the cancer cells have less than two copies of miR-128b DNA per cell.

In some embodiments, quantitative PCR is performed on miR-128b DNA extracted from the biopsy. The forward primer can have at least 50% to 100% sequence identity to SEQ ID NO: 7 and the reverse primer can have at least 50% to 100% sequence identity to SEQ ID NO: 8. In addition, the forward primer can include nucleotides up to 1, 2, 3, 4, or 5 nucleotides upstream or downstream of SEQ ID NO: 7. Similarly, the reverse primer can include nucleotides up to 1, 2, 3, 4, or 5 nucleotides upstream or downstream of SEQ ID NO: 8. Contemplated sequence identities include about 50%, 60%, 70%, 80%, 90%, 95%, and 100% sequence identity to SEQ ID NO: 7 or 8. In other embodiments, quantitative PCR is performed on miR-128a DNA extracted from the biopsy. The forward primer can have at least 50% to 100% sequence identity to SEQ ID NO: 18 and the reverse primer can have at least 50% to 100% sequence identity to SEQ ID NO: 19. In addition, the forward primer can include nucleotides up to 1, 2, 3, 4, or 5 nucleotides upstream or downstream of SEQ ID NO: 18. Similarly, the reverse primer can include nucleotides up to 1, 2, 3, 4, or 5 nucleotides upstream or downstream of SEQ ID NO: 19. Contemplated sequence identities include about 50%, 60%, 70%, 80%, 90%, 95%, and 100% sequence identity to SEQ ID NO: 18 or 19.

In some embodiments, probes are used in measuring the amount of miR-128a or mir-128b DNA relative to an unaffected gene in a cell. Exemplary probes are represented by SEQ ID NO: 11 for miR-128b, SEQ ID NO: 26 for miR-128a, and SEQ ID NO: 12 for CFTR. Any probe that hybridizes to the desired DNA sequence is contemplated, and includes probes of the above-identified sequences having 1, 2, 3, 4, or 5 nucleotides upstream or downstream of those sequences.

It is thus disclosed herein a method of ascertaining responsiveness to treatment of a cancer patient comprising measuring the level of miR-128b or miR-128a in a biopsy obtained from the patient and administering to the patient an EGFR tyrosine kinase inhibitor. The level of miR-128a or miR-128b can be determined by methods known to those skilled in the art. Sometimes, the level of miR-128b is underexpressed relative to normal tissue. A cancer underexpressing miR-128b or miR-128a would be expected to exhibit greater responsiveness to EGFR tyrosine kinase inhibitors. At other times, the level of miR-128b is overexpressed relative to normal tissue. In these instances, the patient can be administered an miR-128b inhibitor or an miR-128a inhibitor with the EGFR tyrosine kinase inhibitor.

It is further disclosed herein a method of treating a cancer patient comprising measuring the number of copies of miR-128b in DNA per cell extracted from a biopsy obtained from the patient and administering to the patient an EGFR tyrosine kinase inhibitor.

Sometimes the ratio of copies of miR-128b to CFTR (or another unaffected gene) will be less than 0.5. A cancer with a ratio of copies less than 0.5 would be expected to exhibit greater responsiveness to EGFR tyrosine kinase inhibitors. At other times, the ratio of copies is 0.5 or greater. In these instances, the patient is further administered a miR-128b inhibitor and/or miR-128a inhibitor with the EGFR tyrosine kinase inhibitor.

In some embodiments, a cancer patient responsive to treatment with an EGFR tyrosine kinase inhibitor can be identified by measuring miR-128a (or miR-128b) levels in a biopsy having cancerous tissue obtained from the patient, and comparing that level to miR-128a (or miR-128b) level in a normal tissue sample. A normal value can be determined by measuring miR-128a (or miR-128b) in normal tissue obtained from the same patient or another individual, or by averaging the level of miR-128a (or miR-128b) in normal tissue taken from a number of individuals.

As discussed below, the cancer can be a lung cancer such as a non-small cell lung cancer (“NSCLC”), including, for example, squamous cell carcinoma, adenocarcinoma, large cell carcinoma, or combinations thereof. It is also contemplated that the methods and compositions described herein are applicable to other EGFR expressing cancers, including but not limited to pancreatic cancer, glioblastoma multiforme, colon cancer, kidney cancer, and bladder cancer. The EGFR inhibitor may be gefitinib or erlotinib. In some embodiments, the EGFR inhibitor is gefitinib.

### Pharmaceutical Compositions

Provided herein are compositions comprising an EGFR tyrosine kinase inhibitor and a miR-128b (or miR-128a) inhibitor. These compositions are used to treat patients having cancer. The cancer can be any form of cancer expressing EGFR, including, but not limited to, pancreatic cancer, cancer, and lung cancer (e.g. NSCLC), for example, squamous cell carcinoma, adenocarcinoma, large cell carcinoma, or combinations thereof. Other NSCLC contemplated herein are pleomorphic, carcinoid tumor, salivary gland carcinoma, and unclassified carcinoma.

Typically, an EGFR expressing cancer is treated with an EGFR tyrosine kinase inhibitor. Gefitinib (N-(3-chloro-4-fluoro-phenyl)-7-methoxy-6-(3-morpholin-4-ylpropoxy)quinazolin-4-amine) and erlotinib (N-(3-ethynylphenyl)-6,7-bis(2-methoxyethoxy)quinazolin-4-amine) are exemplary EGFR tyrosine kinase inhibitors. Other EGFR tyrosine kinase inhibitors include but are not limited to vandetanib, lapitinib, PKI-166. Thus, in some embodiments, the composition comprises gefitinib and a miR-128b inhibitor. Likewise, in some embodiments, the composition comprises erlotinib and a miR-128b inhibitor. Finally, in some embodiments, the composition comprises both gefitinib and erlotinib with a miR-128b inhibitor. In other embodiments, the composition comprises gefitinib and/or erlotinib and a miR-128a inhibitor.

The cancer can further express miR-128b and/or miR-128a. In some embodiments, the cancer is characterized as having a ratio of miR-128b to CFTR DNA greater than 0.5 at the cellular level. In other embodiments, the cancer is characterized as having a ratio of miR-128a to CFTR DNA greater than 0.5 at the cellular level. Any number of approaches can achieve inhibition of miR-128b or miR-128a, for example, a compound can bind to the microRNA and physically interact to inhibit or block its activity or can cause the microRNA to degrade or otherwise prevent it from binding to mRNA. Alternatively, an antagonist which binds the mRNA 3′ UTR can be used to prevent miR-128b from binding, effectively inhibiting the microRNA from suppressing expression of EGFR tyrosine kinase.

Thus, in some embodiments, the miR-128b inhibitor physically interacts with miR-128b. In other embodiments, the miR-128b inhibitor inhibits or blocks the activity of miR-128b. In still other embodiments, the miR-128b inhibitor acts to inhibit miR-128b by preventing it from binding to its 3′ untranslated region binding site on the EGFR mRNA.

In further embodiments, the miR-128a inhibitor physically interacts with miR-128a. In other embodiments, the miR-128a inhibitor inhibits or blocks the activity of miR-128a. In still other embodiments, the miR-128a inhibitor acts to inhibit miR-128a by preventing it from binding to its 3′ untranslated region binding site on the EGFR mRNA.

The miR-128b inhibitor or miR-128a inhibitor can be an antisense nucleic acid molecule, an aptamer, an siRNA, or an RNAi. For example, the miR-128b inhibitor may be a nucleic acid capable of hybridizing to cellular miR-128b RNA. In some embodiments the miR-128b inhibitor may be a nucleic acid capable of hybridizing to cellular miR-128b RNA under stringent hybridization conditions or moderately stringent hybridization conditions. More specifically, the miR-128b inhibitor may be a nucleic acid capable of hybridizing to sequence 20, 21, or 23 in Table 5 below. In another embodiment, miR-128b inhibitor may be a nucleic acid having 60% identity, preferably 65%, 70%, 75%, 80%, 85%, 90%, 91%, 92%, 93%, 94%, 95%, 96%, 97%, 98%, 99%, or higher identity to a nucleic acid that is perfectly complementary to sequence 20, 21, or 23 in Table 5. In other embodiments, miR-128b inhibitor may be a nucleic acid having 60% identity, preferably 65%, 70%, 75%, 80%, 85%, 90%, 91%, 92%, 93%, 94%, 95%, 96%, 97%, 98%, 99%, or higher identity to sequence 1, 2, 11, 24, or 25 in Table 5.

In some embodiments the EGFR tyrosine kinase inhibitor is gefitinib and the miR-128b inhibitor is an oligonucleotide. In other embodiments the EGFR tyrosine kinase inhibitor is erlotinib and the miR-128b inhibitor is an oligonucleotide.

In other embodiments the EGFR tyrosine kinase inhibitor is gefitinib and the miR-128a inhibitor is an oligonucleotide. In still other embodiments the EGFR tyrosine kinase inhibitor is erlotinib and the miR-128a inhibitor is an oligonucleotide.

The elements and characteristics of the pharmaceutical compositions described above are equally applicable to the methods described herein where applicable.

### Methods of Treatment Using Inhibitors

This invention is directed, in part, to methods of treating cancer using EGFR tyrosine kinase inhibitor in combination with a miR-128b inhibitor or a miR-128a inhibitor. In some embodiments, the method comprises administering to a cancer patient an EGFR tyrosine kinase inhibitor and a miR-128b inhibitor or a miR-128a inhibitor. The two inhibitors can be administered in one composition, or can be administered in separate compositions. If separate, the compositions can be administered simultaneously or sequentially. For example, the composition comprising the miR-128b inhibitor can be administered prior to administration of the EGFR tyrosine kinase inhibitor. In any event, the EGFR tyrosine kinase inhibitor and the miR-128b inhibitor or miR-128a inhibitor are administered over the Course of several hours to several months.

As described above, EGFR tyrosine kinase inhibitors include, but are not limited to, gefitinib and erlotinib.

As also described above, the miR-128b inhibitor or miR-128a inhibitor can be an antisense molecule, an aptamer, an siRNA, or an oligonucleotide.

Cancers contemplated for such treatment include those cancers that express EGFR, for example, NSCLC, pancreatic cancer, kidney cancer, colon cancer, glioblastoma multiforme, and bladder cancer. Illustrative NSCLC include, for example, squamous cell carcinoma, adenocarcinoma, large cell carcinoma, and combinations thereof as well as pleomorphic, carcinoid tumor, salivary gland carcinoma, and unclassified carcinoma.

In some embodiments, prior to treatment with the EGFR tyrosine kinase inhibitor and the miR-128b inhibitor or miR-128a inhibitor, the patient can be tested and the cancer identified as potentially responsive to treatment with EGFR tyrosine kinase (see above). This allows a medical provider to tailor a treatment regimen to a particular patient. In some aspects, the method comprises measuring the level of miR-128b or miR-128a in a biopsy obtained from the patient. In other aspects, the method comprises measuring the number of miR-128b DNA copies in a biopsy obtained from the patient. In still further aspects, the method comprises measuring both the level of miR-128b and measuring the number of miR-128b DNA copies in a biopsy obtained from the patient.

### Methods of Treatment Using Mimics

An alternative approach to treatment of cancer expressing EGFR, in some embodiments, is to suppress expression of EGFR. This approach can, in some embodiments, be achieved by administering a miR-128a or miR-128b mimic to a cancer patient. The mimic would have activity similar to that of the miRNA. Thus, provided herein is a method of treating cancer by administering to a cancer patient a composition comprising a miR-128b mimic. Further provided is method of treating cancer by administering to a cancer patient a composition comprising a miR-128a mimic. A mimic can be used to treat cancer alone or in combination with other therapeutic agents, and as such, compositions comprising the mimics in combination with other agents are contemplated herein. Treatment with a mimic of miR-128a or miR-128b will result in down-regulation of EGFR and can initiate further downstream effects that are beneficial in the treatment of cancer.

### Screening Compounds to Identify Cancer Therapeutics

This invention is directed, in part, to methods of identifying cancer therapeutics. In some embodiments, the method comprises screening for compounds that target an miR-128b or miR-128a binding site on the 3′UTR of the EGFR mRNA. In other embodiments, the method comprises screening for compounds that inhibit miR-128b or miR-128a. A compound identified in such manner can be used as a therapeutic in combination with an EGFR tyrosine kinase inhibitor to treat cancer.

In some embodiments, the identified compound is a miR-128a or miR-128b inhibitor. In other embodiments, the identified compound is a miR-128a or miR-128b mimic. Such compounds can be used to treat cancer alone or in combination with other therapeutic agents.

Methods of screening compounds are well known to those skilled in the art. Briefly, tissue culture cells or biopsied cells are treated with a test compound and the effect of this compound on miR-128b or miR-128a levels and/or EGFR levels is measured. Measurements can be attained using Western blot analysis and qRT-PCR for EGFR and qRT-PCR for miR128a and miR128b.

A decrease in miR-128b or miR-128a and/or decrease in EGFR mRNA or protein relative to the baseline or control level after treatment with an inhibitor would indicate that a compound can potentially be used as a cancer therapeutic. A decrease in EGFR after treatment with a potential miR-128a mimic or miR-128b mimic would indicate that the compound can enhance therapy.

### Biomarkers

This invention is directed, in part, to a method for identifying a tissue, a patient, or a patient population predisposed to cancer, for example, NSCLC. The method comprises measuring the level of miR-128b (or miR-128a), the number of miR-128b (or miR-128a) DNA copies, or both, and measuring the level of an unaffected gene across several species such as CFTR, beta-actin, or tubulin in tissue sample obtained from the patient. A tissue sample from a patient predisposed to cancer can exhibit a ratio of miR-128b to CFTR genomic DNA copies less than 0.5 or a ratio of miR-128a to CFTR genomic DNA copies less than 0.5. A tissue sample from a patient predisposed to cancer can exhibit a lower level of miR-128a or miR-128b relative to a standard value obtained from one or more normal control tissues.

### Therapeutic Applications

The pharmaceutical compositions described herein can be administered to a patient in a variety of forms adapted to the chosen route of administration. The compositions can be administered in combination with a pharmaceutically acceptable carrier, adjuvant, or vehicle, and may be combined with or conjugated to specific delivery agents.

In some embodiments, the method comprises administering to an animal (typically a mammal) in need of treatment an effective amount of a composition described herein. In some embodiments, the animal is a human, while in other embodiments, the animal is a mammal other than human. An “effective amount” or “therapeutically-effective amount” means an amount that will achieve the goal of treating the targeted condition.

Suitable formulations and pharmaceutically acceptable carriers or adjuvants suitable for use in such formulations, including fillers, binders, lubricants, stabilizers, aromatic substances, antioxidants, preservatives, dispersing and solubilizing agents, buffers and electrolytes, are known to persons skilled in the art and are described, for example, in standard works such as Sucker et al. (1991), Pharmazeutische Technologie (Pharmaceutical Technology), Deutscher Apotheker Verlag; and Remington (2000), The Science and Practice of Pharmacy, Lippincott, Williams & Wilkins.

The active ingredients in the compositions of this invention can be used in the form of salts derived from inorganic or organic acids. Depending on the particular drug, a salt of the drug may be advantageous due to one or more of the salt's physical properties, such as enhanced pharmaceutical stability in differing temperatures and humidities, or a desirable solubility in water or oil.

Pharmaceutically-acceptable acid addition salts of the drugs used in the compositions described herein may often be prepared from an inorganic or organic acid. Examples of often suitable inorganic acids include hydrochloric, hydrobromic, hydroiodic, nitric, carbonic, sulfuric, and phosphoric acid. Suitable organic acids generally include, for example, aliphatic, cycloaliphatic, aromatic, araliphatic, heterocyclic, carboxylic, and sulfonic classes of organic acids. Specific examples of often suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate(pamoate), ethanesulfonate, benzenesulfonate, pantothenate, 2-hydroxyethanesulfonate, sulfanilate, cyclohexylaminosulfonate, algenic acid, beta-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Pharmaceutically-acceptable base addition salts of the drugs used in the compositions described herein include, for example, metallic salts and organic salts. Preferred metallic salts include alkali metal (group Ia) salts, alkaline earth metal (group IIa) salts, and other physiologically acceptable metal salts. Such salts may be made from aluminum, calcium, lithium, magnesium, potassium, sodium, and zinc. Preferred organic salts can be made from amines, such as tromethamine, diethylamine, N,N′-dibenzylethylenediamine, chloroprocaine, choline, diethanolamine, ethylenediamine, meglumine(N-methylglucamine), and procaine. Basic nitrogen-containing groups can be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), aralkyl halides (e.g., benzyl and phenethyl bromides), and others.

The pharmaceutical formulation can be designed differently as a function of the intended application method. Thus, the pharmaceutical formulation may be adapted, for example, to intravenous, intramuscular, intracutaneous, intrastemal, infusion, subcutaneous, oral, buccal, sublingual, nasal, topical, transdermal, inhalative, rectal, or intraperitoneal administration.

The compositions can be in the form of nasal sprays, creams, sterile injectable preparations, such as sterile injectable aqueous or oleaginous suspensions, or suppositiories.

In some embodiments, a pharmaceutical composition of the invention is orally administered, for example as a capsule, tablet, powder, granulate, pill, suspension, or liquid form. For oral administration as a suspension, the compositions can be prepared according to techniques well-known in the art of pharmaceutical formulation. The compositions can contain microcrystalline cellulose for imparting bulk, alginic acid or sodium alginate as a suspending agent, methylcellulose as a viscosity enhancer, and sweeteners or flavoring agents.

The preferred composition depends on the method of administration. Such compositions may be prepared by a variety of well-known techniques of pharmacy that include the step of bringing into association the active ingredient(s) with one or more excipients. The compositions are often prepared by uniformly and intimately admixing the active ingredient(s) with a liquid or finely divided solid excipient, and then, if desirable, shaping the product. For example, a tablet can be prepared by compressing or molding powder or granules of an active ingredient, optionally with one or more excipients and/or one or more other active ingredients. Compressed tablets can be prepared by compressing, in a suitable machine, the therapeutic agent in a free-flowing form, such as a powder or granules optionally mixed with a binder, lubricant, inert diluent and/or surface active/dispersing agent(s). Molded tablets can be made, for example, by molding the powdered compound in a suitable machine. Formulation of drugs is generally discussed in, for example, Hoover, John E., Remington's Pharmaceutical Sciences (Mack Publishing Co., Easton, Pa.: 1975) (incorporated by reference into this patent). See also, Liberman, H. A., Lachman, L., eds., Pharmaceutical Dosage Forms (Marcel Decker, New York, N.Y., 1980) (incorporated by reference into this patent). See also, Kibbe et al., eds., Handbook of Pharmaceutical Excipients, 3rd Ed., (American Pharmaceutical Association, Washington, D.C. 1999) (incorporated by reference into this patent).

Active ingredients suitable for oral administration may be administered in discrete units comprising, for example, solid dosage forms. Such solid dosage forms include, for example, hard or soft capsules, cachets, lozenges, tablets, pills, powders, or granules, each containing a pre-determined amount of the active ingredient(s). In such solid dosage forms, the active ingredient(s) is ordinarily combined with one or more excipients. If administered with excipients, the active ingredient(s) can be mixed with, for example, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Pharmaceutical compositions particularly suitable for buccal (sub-lingual) administration include, for example, lozenges comprising the active ingredient(s) in a flavored base, usually sucrose, and acacia or tragacanth; or pastilles comprising the active ingredient(s) in an inert base, such as gelatin and glycerin or sucrose and acacia.

Active ingredients suitable for oral administration also can be administered in discrete units comprising, for example, liquid dosage forms. Such liquid dosage forms include, for example, pharmaceutically acceptable emulsions (including both oil-in-water and water-in-oil emulsions), solutions (including both aqueous and non-aqueous solutions), suspensions (including both aqueous and non-aqueous suspensions), syrups, and elixirs containing inert diluents commonly used in the art (e.g., water). Such compositions also may comprise excipients, such as wetting, emulsifying, suspending, flavoring (e.g., sweetening), and/or perfuming agents.

Oral delivery of the therapeutic agents in the present invention may include formulations that provide immediate delivery, or, alternatively, extended or delayed delivery of the active ingredient(s) by a variety of mechanisms. Immediate delivery formulations include, for example, oral solutions, oral suspensions, fast-dissolving tablets or capsules, disintegrating tablets, etc. Extended or delayed delivery formulations include, for example, pH-sensitive release from the dosage form based on the changing pH of the gastrointestinal tract, slow erosion of a tablet or capsule, retention in the stomach based on the physical properties of the formulation, bio-adhesion of the dosage form to the mucosal lining of the intestinal tract, or enzymatic release of the active drug from the dosage form. The intended effect is to extend the time period over which the active drug molecule is delivered to the site of action by manipulation of the dosage form. Thus, in the case of capsules, tablets, and pills, the dosage forms may comprise buffering agents, such as sodium citrate, or magnesium or calcium carbonate or bicarbonate. Tablets and pills additionally may be prepared with enteric coatings. Suitable enteric coatings include, for example, cellulose acetate phthalate, polyvinylacetate phthalate, hydroxypropylmethyl-cellulose phthalate, and anionic polymers of methacrylic acid and methacrylic acid methyl ester.

In some embodiments, the EGFR tyrosine kinase inhibitor and the miRNA-128b inhibitor (or miR128a inhibitor) can be prepared in the same formulation in a mixture. In other embodiments, the EGFR tyrosine kinase inhibitor and the miRNA-128b inhibitor (or miR128a inhibitor) are prepared in separate formulations. In the latter instance, the two separate formulations can be administered together, for example, as a tablet or capsule having part miRNA-128b inhibitor formulation and part EGFR tyrosine kinase inhibitor formulation. The tablet can have an inner core with miRNA 128b inhibitor and an outer layer with the EGFR tyrosine kinase inhibitor formulation. Similarly, capsules can be prepared where any suitable barrier separates the two formulations.

In some instances, it can be desirable to quickly release one active drug, for example, the miRNA-128b inhibitor and subsequently or simultaneously (within about 5 minutes) releasing the second active drug, for example the EGFR tyrosine kinase inhibitor. Any desired timing for release can be achieved by methods of drug formulation known to those skilled in the art.

The compositions described herein can be administered multiple times, with periods typically ranging from once per half hour up to once every 90 days. In typical embodiments, the compositions are administered once per half hour, once per hour, once per 3 hours, once per 5 hours, once per 8 hours, once per 12 hours, once per day, once per 3 days, once per week, or once per 90 days.

Factors affecting the preferred dosage regimen include the type, age, weight, sex, diet, and condition of the patient; the severity of the pathological condition; the route of administration; pharmacological considerations, such as the activity, efficacy, pharmacokinetic, and toxicology profiles of the particular active ingredient used; whether a drug delivery system is utilized; and whether the active ingredient is administered as part of a drug combination. Thus, the dosage regimen actually employed can vary widely, and, therefore, can deviate from the preferred dosage regimen set forth above.

For inhalation or aerosol administration, the compositions can be prepared according to techniques well-known in the art of pharmaceutical formulation. The compositions can be prepared as solutions in saline, using benzyl alcohol or other suitable preservatives, absorption promoters to enhance bioavailability, fluorocarbons, or other solubilizing or dispersing agents known in the art.

For administration as injectable solutions or suspensions, the compositions can be formulated according to techniques well-known in the art, using suitable dispersing or wetting and suspending agents, such as sterile oils, including synthetic mono- or diglycerides, and fatty acids, including oleic acid.

In some embodiments, compositions described herein are administered directly to a target site, such as a tumor. In other embodiments, the compositions are delivered systemically by intravenous injection.

For rectal administration, the compositions can be prepared by mixing with a suitable non-irritating excipient, such as cocoa butter, synthetic glyceride esters or polyethylene glycols, which are solid at ambient temperatures but liquefy or dissolve in the rectal cavity to release the drug.

Alternative pharmaceutical preparations include, for example, infusion or injection solutions, oils, suppositories, aerosols, sprays, plasters, microcapsules and microparticles.

Solutions or suspensions of the compositions can be prepared in water, isotonic saline (PBS) and optionally mixed with a nontoxic surfactant. Alternatively, dispersions can be prepared in glycerol, liquid polyethylene, glycols, DNA, vegetable oils, triacetin, and mixtures thereof. Under ordinary conditions of storage and use, these preparations may contain a preservative to prevent the growth of microorganisms.

The pharmaceutical dosage form suitable for injection or infusion use can include sterile aqueous solutions or dispersions or sterile powders comprising an active ingredient which are adapted for the extemporaneous preparation of sterile injectiable or infusible solutions or dispersions. In all cases, the ultimate dosage form should be sterile, fluid, and stable under the conditions of manufacture and storage. The liquid carrier or vehicle can be a solvent or liquid dispersion medium comprising, for example, water, ethanol, a polyol such as glycerol, propylene glycol, or liquid polyethylene glycols, and the like, vegetable oils, nontoxic glyceryl esters, and suitable mixtures thereof. The proper fluidity can be maintained, for example, by the formation of liposomes, by the maintenance of required particle size, in the case of dispersion, or by the use of non-toxic surfactants. The prevention of the action of microorganisms can be accomplished by various antibacterial and antifungal agents, for example, parabens, chlorobutanol, phenol, sorbic acid, thimerosal, and the like. In some cases, it can be desirable to include isotonic agents, for example, sugars, buffers, or sodium chloride. Prolonged absorption of the injectable compositions can be brought about by the inclusion in the composition of agents delaying absorption such as, for example, aluminum monostearate hydrogels, and gelatin.

Sterile injectable solutions are prepared by incorporating the compounds in the required amount in the appropriate solvent with various other ingredients as enumerated above, and, as required, followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

The compositions described herein can also be included in a combination therapy for simultaneous or sequential administration depending on the type and severity of the disease to be treated.

For example, a sales unit containing an EGFR tyrosine kinase inhibitor and a miR-128b inhibitor may contain a further active ingredient (or several further active ingredients). In this case, the compounds may be present in a single pharmaceutical formulation, for example a combination tablet, or in different application units, for example in the form of two or three separate tablets. Depending on need, the active ingredients can be administered simultaneously or at separate times.

In a combination preparation, a sequential administration can be achieved, for example, by using a form of administration, for example an oral tablet, having two or more zones, e.g., layers, with a differing release profile for pharmaceutically active components. It will be clear to the person skilled in the art that in the context of the present invention, various forms of administration and application patterns are conceivable which are all the subject of the invention.

One embodiment of the invention therefore relates to a pharmaceutical composition which comprises an EGFR tyrosine kinase inhibitor and a miR-128b inhibitor along with an additional active ingredient for simultaneous or sequential administration to a patient. The additional active ingredient for simultaneous or sequential administration can be, for example, an active ingredient for treating cancer-associated pain, an anti-emetic, or a further agent for treating the basic disease.

Within the application, unless otherwise stated, the techniques utilized may be found in any of several well-known references, such as: Molecular Cloning: A Laboratory Manual (Sambrook et al. 1989 Molecular Cloning: A Laboratory Manual), Gene Expression Technology (Methods in Enzymology, Vol 185, ed. D. Goeddel, 1991 Academic Press, San Diego, Calif.), “Guide to Protein Purification” in Methods in Enzymology (M. P. Deutshcer, 3d. 1990 Academic Press, Inc.), PCR Protocols: A Guide to Methods and Applications (Innis et al. 1990 Academic Press, San Diego, Calif.), Culture of Animal Cells: A Manual of Basic Technique, 2^{nd} ed. (R. I. Freshney 1987 Liss, Inc, New York, N.Y.), and Gene Transfer and Expression Protocols, (pages 109-128, ed. E. J. Murray, The Humana Press Inc., Clinfton N.J.

### Kits and Assays

This invention is directed, in part, to a kit for use in identifying a cancer patient responsive to treatment with an EGFR tyrosine kinase inhibitor. The method of identifying such a patient is substantially the same as described above. In some embodiments, the kit comprises control DNA, control forward and reverse primers, control probe, and forward and reverse miR-128b primers, and miR-128b probe. In other embodiments, the kit comprises miR-128a primers and probe in addition to or in place of the miR-128b primers and probe. The kit can optionally comprise any reagents needed to perform quantitative PCR, and/or instructions for performing any methods described herein.

This invention also is directed, in part, to a kit comprising the compositions described herein. In some embodiments, compositions described herein are provided in the kit. As described in the above Pharmaceutical Composition section, the compositions can comprise EGFR tyrosine kinase inhibitor, miRNA-128a inhibitor, and/or miRNA-128b inhibitor. The kit is used to treat a cancer in an animal. In some embodiments, the animal is a mammal. In some such embodiments, the mammal is a human. In some embodiments, the disease is cancer, for example, lung cancer. In other embodiments, the disease is NSCLC.

In some aspects, the compositions are provided with a means for administration.

In further aspects, the kit comprises instructions for, for example, using the kit.

### EXAMPLES

The following examples are merely illustrative, and not limiting to this disclosure in any way.

### Materials and Methods

### Bioinformatics

Public-access databases (Sanger, TargetScan, Emsembl, and UCSC Genome Browser) were utilized to determine miRs associated with EGFR, determine chromosomal locations for EGFR and its predicted regulatory miRs based on 3′UTR binding sites. MiR binding predictions were confirmed by manually analyzing the EGFR 3′UTR and mature miR sequences.

### MiR-128b Mimic and Inhibitor

A mimic of miR-128b was purchased from Dharmacon (C-300139-01-0010, Boulder, Colo.) and an inhibitor (anti) of miR-128b was purchased from Ambion (17000, Foster City, Calif.). Both the mimic and inhibitor were oligonucleotides.

### Primer Design

The genomic DNA sequences of EGFR 3′ UTR and miR-128b were obtained from the human genome assembly (http://www.ensembl.org). The GeneFisher internet tool was used to design primers sufficient to encompass the desired genomic DNA product. (http://bibiserv.techfak.uni-bielefeld.de/cgi-bin/gf_submit?mode=STARTUP&qid=na& sample=dna)

### PCR and Sequencing Methods for Genomic DNA

Genomic DNA was prepared from cell lines using the Qiagen DNeasy Tissue kit (69504, Qiagen, Valencia, Calif.).

Touch Down PCR was used with GoTaq Green Master Mix (Promega, Madison, Wis.) with each reaction containing 1 μL of genomic DNA as a template, an activation step of 95° C. for 2 minutes, then denaturation at 94° C. for 30 seconds; annealing starting at 63° C. and stepping down by half degrees until 53° C. for 1 minute, and extension at 72° C. for 1 minute. An additional 15 cycles was performed at 55° C. A final 10 minute extension at 72° C. was performed following completion of the cycles. The amplified PCR products were electrophoresed on 1.5% gel visualized with ethidium bromide and a UV light source. PCR product bands were excised and purified using the Qiaquick Gel Extraction Kit (28704, Qiagen, Valencia, Calif.). Purified PCR products were quantified using a ND-1000 (NanoDrop, Wilmington, Del.) spectrophotometer, and then sequenced by the University of Colorado Cancer Center DNA Sequencing Core using both forward and reverse primers with an ABI 3730 DNA Sequencer and ABI BigDye Terminator kit 1.1v (ABI, Foster City, Calif.) according to the manufacturer's instructions. Two reviewers manually reviewed the forward and reverse chromatograms using Chromas Lite 2.01 (Technelysium Pty, Tewantin Qld, Australia). Alignments and mutation analysis were performed using BLAST (National Center for Biotechnology Information) software.

TABLE 1
Primers for genomic DNA

| | | Primer |
|---|---|---|
| 3p22 encompassing miR-128b | forward | 5′-AGGTACAAGAAGGTGAAGCA-3′ |
| | | (SEQ ID NO: 1) |
| 3p22 encompassing miR-128b | reverse | 5′-GATGTCTGTGATTGGTGCTA-3′ |
| | | (SEQ ID NO: 2) |
| EGFR 3′UTR binding site 1 | forward | 5′-ATTAGCTCTTAGACCCACAGACT |
| | | GG-3′ (SEQ ID NO: 3) |
| EGFR 3′UTR binding site 1 | reverse | 5′-TTCTTGCTGGATGCGTTTCTGTAA |
| | | AT-3′ (SEQ ID NO: 4) |
| EGFR 3′UTR binding site 2 | forward | 5′-TACCCTGAGTTCATCCAGGCC-3′ |
| | | (SEQ ID NO: 5) |
| EGFR 3′UTR binding site 2 | reverse | 5′-AGTGGAAGCCTTGAAGCAGAAC-3′ |
| | | (SEQ ID NO: 6) |

### Cell Culture

The NSCLC cell line, NCI-H 157, was provided by Drs. John Minna and Adi Gazdar (University of Texas Southwestern Medical School, Dallas, Tex.). The NSCLC lines A549, Colo699, and NCI-H520 were obtained from the American Type Culture Collection (Rockville, Md.). The NCI-H358 line was obtained from Dr. Isaiah J. Fidler (University of Texas M.D. Anderson Cancer Center, Houston, Tex.). The H3255 cell line was a gift from Dr. Bruce Johnson (Dana-Farber Cancer Center, Boston, Mass.). All cell lines (referred to herein as H157, A549, Colo699, H520, H358, and H3255) were maintained in RPMI media supplemented with 10% heat-inactivated fetal bovine serum (Hyclone, Logan, Utah) in a humidified incubator with 5% CO₂.

Growth Inhibition of NSCLC Cells by miR-128b Mimic or Inhibitor Alone or in Combination with Either Gefitinib or Cetuximab.

Gefitinib was provided by Astra-Zeneca Pharmaceuticals and Cetuximab was provided by ImClone Systems, Inc. (New York, N.Y.). Gefitinib stock solutions were prepared in DMSO and stored at −20° C. Cetuximab stock solution was supplied at a concentration of 2 mg/mL and formulated in a preservative-free solution containing 8.48 mg/mL sodium chloride, 1.88 mg/mL sodium phosphate dibasic heptahydrate, 0.41 mg/mL sodium phosphate monobasic monohydrate, and water. Prior to use, drug stocks were diluted in fresh media. The growth inhibitory effects of miR-128b mimic at 4 nM (Dharmacon, Lafayette, Colo.) and miR-128b inhibitor at 4 nM (Ambion, Austin Tex.) alone or in combination with gefitinib or cetuximab were evaluated using a modified tetrazolium salt (MTT) assay (Carmichael et al. 1988 Br. J. Cancer 57: 540). Cells were seeded in 96-well flat bottomed plates (Corning Inc., Corning, N.Y.) in 50 μL RPMI media supplemented with 10% heat-inactivated fetal bovine serum followed by transfection with miR-128b mimic or inhibitor (HiPerfect Transfection Reagent, Qiagen, Valencia, Calif.) at least 6 hours after seeding to bring total volume to 100 μL. Following an overnight incubation, varying concentrations of gefitinib (range 0.1-15 μM) or cetuximab (range 25-100 nM) were added to control, mimic, or inhibitor treated cells for an additional 72 hour incubation. An absorbance at 490 nm of 0.1-0.4 was sought. The optimum numbers of cells seeded to achieve this range were determined to be 5,000 cells for A549, H358, and H157 cell lines, and 5,000 to 7,500 cells for H3255, H520, and Colo699 cell lines. No IC₅₀ growth inhibition was observed in these tested cell lines with cetuximab alone at concentrations up to 100 nM (Raben et al. 2005 Clin. Cancer Res. 11: 795). Tetrazolium salt was added at a concentration of 0.4 mg/mL to each well following the 72 hour incubation. The plates were then incubated with the salt for 4 hours at 37° C. At 4 hours, the medium was aspirated off, leaving the dark blue formazan product at the bottom of the wells. The reduced MTT product was solubilized by adding 100 μL of 0.2 N HCl in 75% isopropanol and 23% MilliQ water to each well, then mixed thoroughly with a multichannel pipetter. The absorbency of each well was measured using an automated plate reader (Molecular Devices, Sunnyvale, Calif.). MTT with mimic co-transfection was performed in duplicate or triplicate, while inhibitor co-transfection was performed once as no discernable difference was measured.

### Antibodies and Western Blotting

NSCLC cells were seeded at 3×10⁵ to 4×10⁵ cells per 60 mm plate and transfected with 4 nM miR-128 inhibitor or 4 nM miR-128b mimic using HiPerfect Transfection Reagent according to manufacturer's instructions (Qiagen, Valencia, Calif.), followed by a 48 hour incubation. Molecular weight markers (Bio-Rad) were loaded to ensure proteins of interest were at the appropriate size. Cells were lysed and cellular lysates were separated on NuPage 4-12% BisTris Gels (NP0323BOX, Invitrogen, Carlsbad, Calif.) and transferred to polyvinylidene difluoride paper (1380131, Invitrogen, Carlsbad, Calif.). Membranes were probed with primary antibodies in PBS-2% nonfat dry milk powder followed by incubation with appropriate horseradish peroxidase-conjugated secondary antibody in PBS-2% nonfat dry milk powder. Immunoblots were developed with Supersignal West Femto Maximum Sensitivity Substrate (34096, Pierce, Rockford, Ill.) and analyzed using a Chemi-Doc chemoluminescence detector (Bio-Rad, Hercules, Calif.), except total EGFR was originally developed with Millipore Immobilon Western Chemiluminescent HRP Substrate (Millipore WBKLSO100 Billerica, Mass.).

The following antibodies were used: anti-EGFR antibody and anti-phospho-EGFR (Tyr1068) antibody (2232 and 2234, Cell Signaling Technology, Beverly, Mass.); anti-GFP antibody-2 and Pan actin Ab-5 (MS-1315-P1 and MS-1295-P1, Neomarkers, Fremont, Calif.); and horseradish peroxidase-conjugated donkey anti-rabbit IgG or horseradish peroxidase-conjugated sheep anti-mouse IgG (NA934V and NXA931, Amersham Biosciences, Buckinghamshire, England). The primary antibodies were used at a 1:1,000 dilution and the secondary antibodies used at 1:10,000 dilution.

### Densitometric Analysis

Autoradiographs of immunoblots were scanned with a Bio-Rad Chemi Doc system using Quantity One (version 4.1) software (Bio-Rad Laboratories, Hercules, Calif., USA). The Chemi Doc system features an 8-bit CCD camera with a ½″ array and an 8 mm to 48 mm zoom lens for high-resolution digital images. Bands of interest were measured and quantified with normalization with background intensity. Each immunoblot was normalized to the cell line's control. Bands of interest were then normalized to their specific actin band intensity. Calculations of relative intensity and normalization were performed using Microsoft Excel 2002 (Microsoft Corporation, Redmond, Wash.).

### G-Banding and SKY

Cells in culture were blocked in metaphase with colcemid (0.05 μg/ml) prior to hypotonic swelling in a 4:1 mixture of 0.075M KCl and 1% sodium citrate. Cells were fixed using 3:1 methanol and glacial acetic acid. Slides were prepared, incubated overnight at 60° C. and then submitted to GTL-banding technique following standard procedures, including 20-25 second incubation in trypsin and 2-3 min staining with Leishman's stain (van Bokhoven et al. 2003 The Prostate 57: 226). Metaphase chromosomes were digitally imaged and karyotyped with the Genus workstation (Applied Imaging Corp-AI, Santa Clara, Calif.). Spectral karyotyping (SKY) was performed with reagents and equipment from Applied Spectral Imaging (ASI, Vista, Calif.) according to protocol published elsewhere (van Bokhoven et al. 2003 The Prostate 57: 226). Image acquisition was performed using the SD200 Spectracube coupled to an Olympus BX60 epifluorescence microscope, a custom designed optical filter (SKY-1, Chroma Technology Corp, Rockingham, Vt.), and the SpectralImaging v2.6 software. Analysis was performed using SKYView v2.1. At least 10 metaphase spreads were completely karyotyped for each cell line and abnormalities were interpreted according to the ISCN 2005 guidelines (Shaffer and Tommerup, Eds. 2005 An International System for Human Cytogenetic Nomenclature, S. Karger, Basel, Switzerland). Chromosome breakpoints were assigned based on the SKY-inverted DAPI images and the G-banding results.

### Fluorescence In Situ Hybridization (FISH)

Dual-color FISH assays with the EGFR-SpectrumOrange/CEP7-SpectrumGreen probe set (Vysis/Abbott Molecular, Des Plaines, Ill.) were performed per protocol previously published (Helfrich et al. 2006 Clin. Cancer Res. 12: 7117). Following dehydration, cells attached to the slides were incubated for 5 minutes in pepsin (0.01% in 0.01 M HCl) at 37° C. and fixed in 1% formaldehyde at room temperature for 10 minutes. The EGFR/CEP probe was applied according to the manufacturer's instructions, and codenaturation of probe and target DNAs was achieved by incubation at 80° C. for 6 minutes. Hybridization was allowed to occur at 37° C. for 20 hours, and the unbound probe was washed out in three incubations in 50% formamide/2×SSC and one incubation in 2×SCC/0.1% NP40, each for 6 minutes at 46° C. Chromatin was counterstained with 4′,6-diamidino-2-phenylindole (DAPI) in VECTASHIELD® antifade (Vector Lab, Burlingame, Calif.). At least 20 metaphase cells and 200 interphase cells were analyzed per cell line using epifluorescence microscopes coupled with triple (blue/red/green) and single band filters for blue, red, and green (Chroma Technology Corp., Rockingham, Vt.). Images were acquired using cooled CCD camera and merged by CytoVysion software (AI).

### Comparative Genomic Hybridization (CGH)

DNA from cell lines and normal specimens (one female and one male) used as reference was extracted by standard procedure. Aliquots of tumor and normal (used as control) DNAs were labeled with SpectrumRed dUTP (SR) using nick translation (Vysis/Abbott Laboratories, Des Plaines, Ill., USA); aliquots of normal DNA used as reference were labeled with Spectrum Green dUTP (SG). The SR-labeled DNA (tumor or normal) and the SG-labeled reference DNA were combined in a ratio of 1:1.5, respectively, and competitively hybridized to normal metaphase spreads (Kallioniemi et al. 1992 Science 258: 818). Post hybridization washes included 2-min incubation at 74° C. in 0.4×SSC/0.3% NP-40, and 1-min incubations at room temperature in 2×SSC/0.1% NP-40 and in 2×SSC. Slides were counterstained with DAPI in VECTASHIELD® Mounting Medium (Vector Laboratories, Burlingame, Calif.). Each CGH assay included a control slide hybridized with SR-labeled normal DNA and SG-labeled reference DNA.

Slides were examined using epifluorescence microscopy: 5 metaphases from each control and 10-15 metaphases for each cell line were imaged and karyotyped using the PathVysion software (Applied Imaging, Santa Clara, Calif.). The intensities of light emitting red and green detected by the imaging system were fed into logarithmic equations and the respective values were plotted to produce the graphical representation of gene gain and loss as seen in the CGH profiles. An excess of red light indicated genomic gene gain for the SR-labeled DNA, while an excess of green light indicated genomic loss for the SR-labeled DNA. Ratio values of variance 1.15 and 0.85 were used as definitions for gene gain and loss, with a standard of 1. For each cell line, individual metaphase profiles were combined to create a master profile. Abnormalities that did not occur consistently were assumed to be the result of inherent genomic instability of cancer cells rather than clonal accumulation and were removed from the profile to minimize statistical noise. Regions consistently shown to harbor random deviations in CGH such p-arms of acrocentric chromosomes, telomeric and centromeric regions (Kallioniemi et al. 2004 Genes Chromosomes Cancer 10: 231) were not included in the analyses.

### Tumor Sample Microdissection

After appropriate approval from the institution and written informed consent for comprehensive use of molecular and pathologic analysis was received from each patient, NSCLC specimens were formalin-fixed, paraffin-embedded at Tokyo Medical University, Tokyo, Japan. Information on gender, age, histology, cigarette use, response, and survival was obtained for each patient. Tumor specimens were microdissected under stereoscopic microscopy (LEICA MZ12, Leica Microsystems, Wetzlar, Germany). DNA was extracted from tumor cells with the DNeasy Tissue Kit (Qiagen, Valencia, Calif.).

### Statistical Analysis

For all tests, a level of P<0.05 was considered statistically significant. Fisher's exact test for count data was used to analyze proportions among the factors studied. To determine which factors had an influence on response to gefitinib, logistic regression was performed. In this case, response or stable disease was considered a positive outcome while progressive disease was considered negative. The Kaplan-Meier method was used to estimate the probability of survival as a function of time. Survival was calculated from the date of first gefitinib treatment to the date of death from any cause; all other patients were censored at the time of their last follow-up. Significant differences between survival curves were analyzed using the log-rank test. Multivariate analysis of the relative importance of the factors to survival was performed using the Cox proportional-hazards method. Correlation co-efficients were used to determine the correlation between EGFR and miR-128b expression values above and below gefitinib IC₅₀. All calculations were performed using R statistical software (http://crans-projectorg/).

### DNA Quantitative PCR

A standard curve was created by amplifying genomic DNA from H157 line using Touch Down PCR with GoTaq Green Master Mix as described above with the following Taqman primers for the miR-128 DNA locus:

Forward miR-128b primer:
(SEQ ID NO: 7)
5′-GCCGATACACTGTACGAGAGTGA-3′
Reverse miR-128b primer:
(SEQ ID NO: 8)
5′-GGAGTGTGACACAGTAGGGAAAGA-3′;

primers for the miR-128a DNA locus:

Forward miR-128a primer:
(SEQ ID NO: 24)
5′-GGGCCGTAGCACTGTCTGA-3′
Reverse miR-128a primer:
(SEQ ID NO: 25)
5′-CCAGGAAGCAGCTGAAAAAGA-3′

and forward and reverse CFTR primers (Fortna et al. 2004 PLoS Biol. 2: E207) as the standard reference gene:

Forward CFTR primer:
(SEQ ID NO: 9)
5′-CGCGATTTATCTAGGCATAGGC-3′
Reverse CFTR primer:
(SEQ ID NO: 10)
5′-TGTGATGAAGGCCAAAAATGG-3′.

Amplified PCR products were electrophoresed and DNA product was isolated and concentration was determined as described above. Copies per μL were determined by multiplying the product (ng/μL) by 1×10⁻⁹ divided by the product of (# PCR product base pairs×660 g/mole)/(6.023×10²³ copies). The DNA product was then diluted serially to 6-8 dilutions ranging from 5×10¹⁰ to zero copies. DNA copy number was determined by conversion of C(t) to copies per μL against the standard curves of miR-128b and CFTR. Taqman PCR was carried out with tumor sample and cell line DNA using the above primers for miR-128b and CFTR and the following probe sequences:

miR-128b
(SEQ ID NO: 11)
5′-6FAM-TAGCAGGTCTCACAGTGAACCGGT-3′-TAMRA
miR-128a
(SEQ ID NO: 26)
5′-TTTACATTTCTCACAGTGAACCGGT-3′-TAMRA
CFTR
(SEQ ID NO: 12)
5′-VIC-TGCCTTCTCTTTATTGTGAGGACACTGCTCC-3′-TAMRA.

Each sample was analyzed in triplicate on a quantitation run. The ratio of miR-128b to CFTR DNA copies was determined. Samples with a ratio of ≦0.5 were considered to have a deletion of DNA (also referred to herein as a loss of heterozygosity (i.e. LOH)) at the miR-128b locus. Cell line results were normalized to the lowest ratio (H3255) to determine relative DNA copy number.

Quantitative Reverse Transcription PCR of miR-128b

Using Applied Biosystems Taqman system, RT was carried out, followed by quantitation against RNAU6B control according to manufacturer's instructions. Each sample was analyzed in triplicate on each quantitation run. Cell line results were normalized to the lowest ratio (H3255) to determine relative miR-128b expression.

### GFP EGFR 3′UTR Reporter Construction and Expression

The 3′UTR of EGFR is encoded in exon 28. Genomic DNA from H157 was amplified using GoGreen Taq (Promega, Madison, Wis.).

The forward primer sequence (forward primer-EGFR-3′UTR binding site 1):

5′-ATTAGCTCTTAGACCCACAGACTGG-3′ (SEQ ID NO: 3) and the reverse primer sequence (reverse primer-EGFR-3′UTR binding site 2): 5′-AGTGGAAGCCTTGAAG CAGAAC-3′ (SEQ ID NO: 6) were used. The PCR product was purified using the PCR clean up kit Qiagen (28106, Qiagen, Valencia, Calif.) and inserted into the Topo T-Vector cloning kit (K4500-01SC, Invitrogen, Carlsbad, Calif.). A T-vector clone with the EGFR-3′UTR correctly orientated was isolated and restricted with Xho1 and BamHI sites in the pTopo2.1 T-vector encompassing the EGFR-3′UTR fragment. This fragment was ligated into pEGFP-C1 (Clontech Laboratories, Mountain View, Calif., USA) at the Xho1 and BamHI sites. A549, H157, H358, H520, and Colo699 cells were then transfected using the lipophillic reagent Effectene Transfect Reagent (301427, Qiagen, Valencia, Calif.) with either the empty vector GFP and GFP-EGFR-3′UTR construct at a concentration of 1000 ng of transfection product. Following 48 hours of incubation, GFP protein was quantitated by Western blot (described in Antibodies and Western blotting) and mRNA was quantitated by qRT-PCR (described in qRT-PCR). Equal transfection was confirmed by GFP quantitation as described below.

### GFP Quantitation

DNA copy of GFP using the following primers was performed to determine transfection equivalence between controls and cell line treatment conditions:

GFP forward
(SEQ ID NO: 13)
5′-CGACAAGCAGAACAACGGCATCAA-3′
GFP reverse
(SEQ ID NO: 14)
5′-AACTCCAGCAGGACCATGTGA-3′.

Transfection plasmids contain cDNA which was amplified by the primers. Quantitative PCR was performed in triplicate on a quantitation run using the Applied Biosystems SYBR Green PCR kit according to the manufacturer's instructions. The ratio of GFP DNA of experimental conditions to GFP control for each cell line transfected was determined. Ratios of 0.8-1.2 were considered equivalent transfection of GFP constructs.

Relative GFP mRNA expression was determined using extracted RNA. The GFP primers (above) and the beta-actin primers (below), serving as internal controls, were used after a reverse transcription reaction with the Applied Biosystems High Capacity Reverse Transcription kit:

Beta-actin forward
(SEQ ID NO: 15)
5′-ATCCACGAAACTACCTTCAACTC-3′
Beta-actin reverse
(SEQ ID NO: 16)
5′-GAGGAGCAATGATCTTC-3′

Quantitative PCR was performed in triplicate on a quantitation run using the Applied Biosystems SYBR Green PCR kit according to the manufacturer's instructions. Relative GFP mRNA expression levels were compared between each cell line's GFP and GFP EGFR 3′UTR transfection conditions.

### EGFR Immunohistochemistry

NSCLC cell lines and tumor slides were collected and stained with anti-EGFR antibody (anti-EGFR clone 31G7, Zymed, San Francisco, Calif.), as previously described by Helfrich et al. and Hirsch et al. (2006 Clin. Cancer Res. 12: 7117; 2003 J. Clin. Oncol. 21:

3798). Cell line specimens were scored by the dominant intensity pattern of staining (1, negative or trace; 2, weak; 3, moderate; 4, intense). An EGFR IHC intensity scoring system was applied to patient tumor samples (Hirsch et al. 2003 J. Clin. Oncol. 21: 3798). All grading was performed by a board-certified pathologist (W.A.F).

### Results

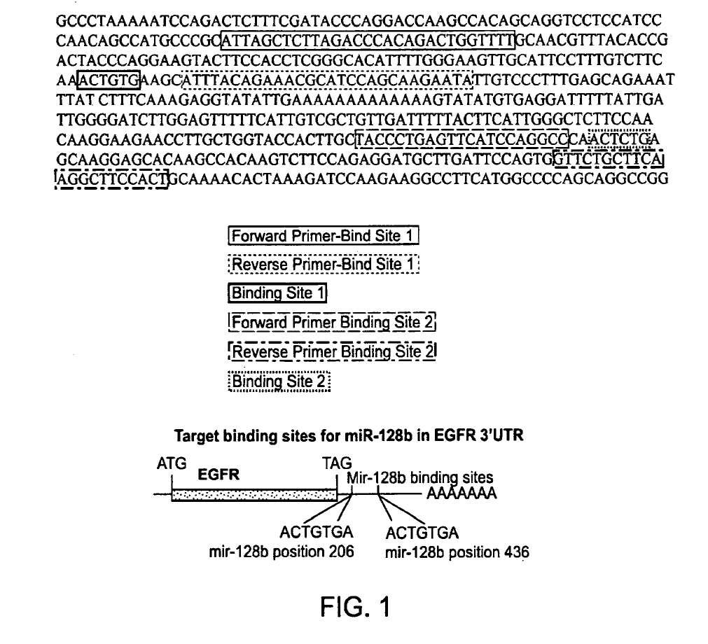

Using public-access resources (Supplementary Section-Bioinformatics), miR-128b was studied as a potential EGFR regulator. Potential miR binding sites on the EGFR-3′ untranslated region (3′UTR) (FIG. 1) were identified.

Cytogenetic analysis on five NSCLC lines (H157, A549, H520, H358, and H3255) by G-banding, spectral karyotyping (SKY) and comparative genomic hybridization (CGH), was performed as an initial screen. In addition, two potential binding sites and the chromosome 3p22 region that encompasses miR-128b in these five lines were amplified and sequenced. By SKY and CGH, there were losses and/or rearrangements involving 3p22 in four of five lines (Table 2). By PCR and DNA sequencing, only H3255 did not have the miR-128b containing amplicon in 3p22. This cell line is well-characterized by its L858R EGFR mutation (Paez et al. 2004 Science 304: 1497) and is strongly positive for EGFR (4+ on a 1 to 4 scale [Hirsch et al. 2003 J. Clin. Oncol. 21: 3798]) by IHC and Western blot. No additional mutations were detected in amplicons in the predicted EGFR-3′UTR binding sites or the 3p22 region in the remaining lines.

TABLE 2
Cell Line Karyotype, miR-128b Quantitation, and EGFR IHC Expression

| | | Relative | Relative | FISH 7p | EGFR Protein |
|---|---|---|---|---|---|
| Cell | 3p22 chromosomal | miR-128b | miR-128b | (EGFR) | Expression |
| Line | status | DNA copy | RNA Expression | copy Number | by IHC |
| H157 | Balanced by CGH; | 104.3 | 15.3 | 3.1 | 4+ |
| | breakpoint at 3p21- | | | | |
| | 22 by SKY | | | | |
| H358 | 3p loss by CGH, | 20.7 | 41.3 | 3 | 4+ |
| | breakpoint at 3p22 | | | | |
| | by SKY | | | | |
| A549 | Balanced by CGH; | 55.1 | 35.4 | 2.5 | 4+ |
| | breakpoint at 3p21- | | | | |
| | 22 by SKY | | | | |
| H3255 | 3p loss by CGH; | 1* | 1† | 20 | 4+ |
| | unaffected by SKY | | | | |
| H520 | Balanced by CGH; | 151 | 12,200 | 2.65 | 1+ |
| | unaffected by SKY | | | | |
| BLE1 | N/A | N/A | 1.2 × 10⁶ | N/A | Limited to |
| | | | | | Basal Layer‡ |
| BLE2 | N/A | N/A | 1.9 × 10¹⁷ | N/A | Limited to |
| | | | | | Basal Layer‡ |

Key:
BLE—Benign lung epithelium
N/A—Not available
*H3255 required up to 45 PCR cycles to observe a value for DNA copy. The ratio of DNA miR-128b to CFTR was 0.002 indicating deletion. Relative DNA copy of other lines was normalized to this value. The remaining NSCLC had ratios ≦0.5 indicating some degree of DNA copy deletion at the miR-128b locus.
†H3255 required up to 45 PCR cycles to observe a value. In order to derive relative expression levels among the cell lines, H3255 was set to 1; however, it is probable that no expression of miR- 128b is present, especially with the determination of DNA copy.
‡Normal bronchial lung tissue

Relative expression of miR-128b was determined by qRT-PCR (Table 2). The H520 line (negative for EGFR by IHC, 1+) has more miR-128b expression (by a factor of 295) than the other four lines, all of which have strong staining intensity for EGFR (4+). These four cell lines (H157, H358, A549, and H3255) were determined to have a 3p loss by CGH and/or rearrangements with a breakpoint at 3p21-p22 by SKY (Table 2). In addition, DNA copy number of miR-128b locus was determined by quantitative PCR (Fortna et al. 2004 PLoS Biol. 2: E207), showing relative differences in DNA quantity (range 1-151) compared to H3255 (Table 2), though all lines were determined to have some degree of miR-128b locus deletion. There is clearly a marked difference in miR-128b expression levels between NSCLC cell lines, with relative expressions ranging from 1 to 12,200. Furthermore, RNA from two benign lung epithelium lines (BLE) had greater relative miR-128b expression level than H520 (factor of at least 98), suggesting that high levels of miR-128b message are required to suppress EGFR levels.

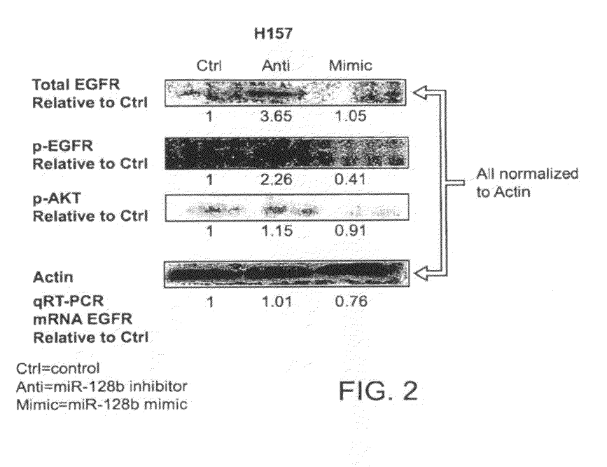

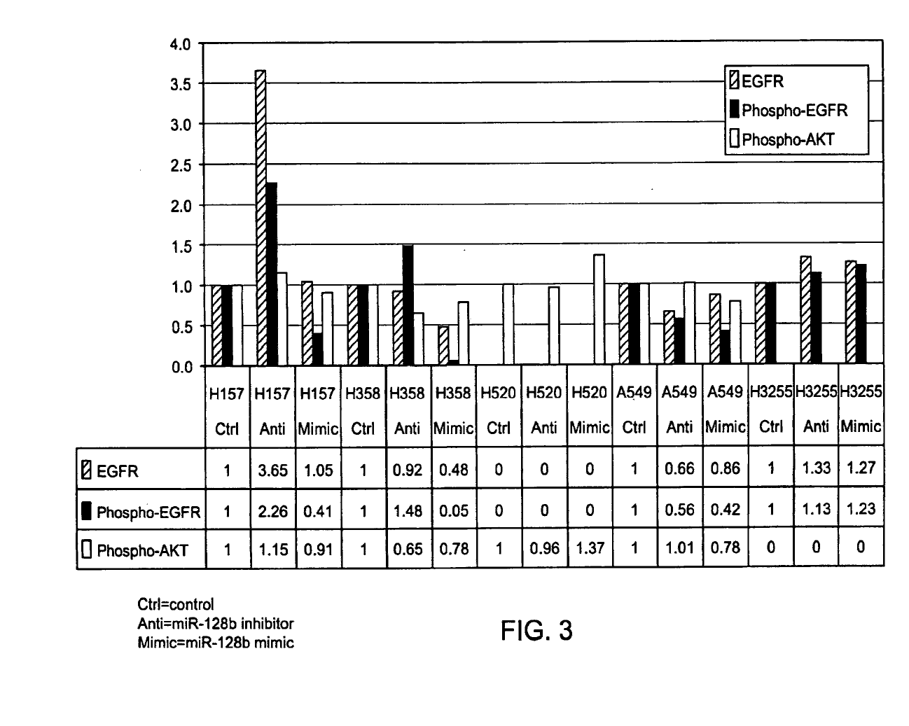

To determine whether miR-128b regulates EGFR, cells were treated with miR-128b mimic or inhibitor at 4 nM for 48 hours. For EGFR expressing cell lines, inhibitor treatment resulted in upregulation of EGFR (2 of 4) and p-EGFR (3 of 4) protein, while mimic treatment resulted in downregulation of EGFR (2 of 4) and p-EGFR (3 of 4) protein by Western blot (FIGS. 2 and 3). Relative EGFR mRNA compared to control was upregulated in 1 of 5 lines with inhibitor treatment and downregulated in 4 of 5 with mimic treatment. These results demonstrate that miRs can either lead to degradation of EGFR message or inhibit EGFR protein translation with different effects that are cell line specific. In addition, miR-128b initiates downstream effects by altering p-AKT by Western blot (FIGS. 2 and 3).

After treatment with gefitinib above and below each cell line's IC₅₀, miR-128b expression levels were altered. Relative EGFR mRNA and miR-128b expression levels were positively correlated (r=0.91) (Table 3). It is clear that treatment of cells with EGFR-TKI alters both EGFR mRNA and miR-128b expression. This phenomenon has been observed with other miRs in cholangiocarcinoma lines after treatment with gemcitabine (Meng et al. 2006 Gastroenterology 130: 2113). Change in a particular miR after cellular stress can alter cell viability or proliferation potential (Meng et al. 2006 Gastroenterology 130: 2113; Xu et al. 2003 Current Biol. 13: 790; Ambros 2003 Cell 113: 673). Additionally, treatment with both gefitinib and miR-128b mimic or inhibitor was explored for synergism in reducing the IC₅₀. No significant changes were observed, possibly due to additional downstream regulatory effects of miR-128b.

TABLE 3
EGFR/miR-128b qRT PCR after Gefitinib
Below/Above IC₅₀ at 72 hours

| Cell Line and Target | Control | <IC₅₀ | >IC₅₀ |
|---|---|---|---|
| H157 EGFR | 1.0 | 0.5 | 0.5 |
| H157 miR-128b | 1.0 | 0.1 | 2.5 |
| A549 EGFR | 1.0 | 1.3^{‡} | 0.7^{γ} |
| A549 miR-128b | 1.0 | 0.0 | 6.4 |
| Colo699 EGFR | 1.0 | 0.9 | 1.2 |
| Colo699 miR-128b | 1.0 | 0.0 | 0.0 |
| H358 EGFR | 1.0 | 1.3 | 1.6 |
| H358 miR-128b | 1.0 | 1.5 | 0.5 |
| H3255 EGFR | 1.0 | 1.1 | 0.8 |
| H3255 miR-128b | 1.0 | 2.7 | 30.9 |
| H520 EGFR | 1.0 | 1.6 | 3.9 |
| H520 miR-128b | 1.0 | 25.1 | 157.0 |

*miR-128b and EGFR are correlated when > IC₅₀
^{‡}r = 0.64
^{γ}r = 0.91

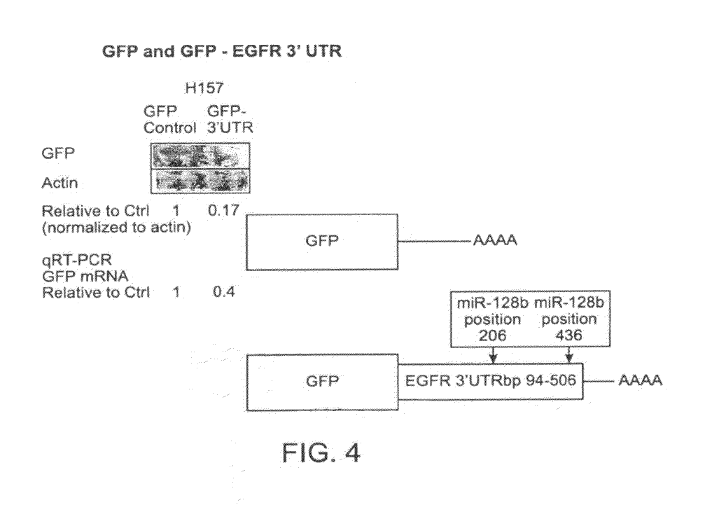

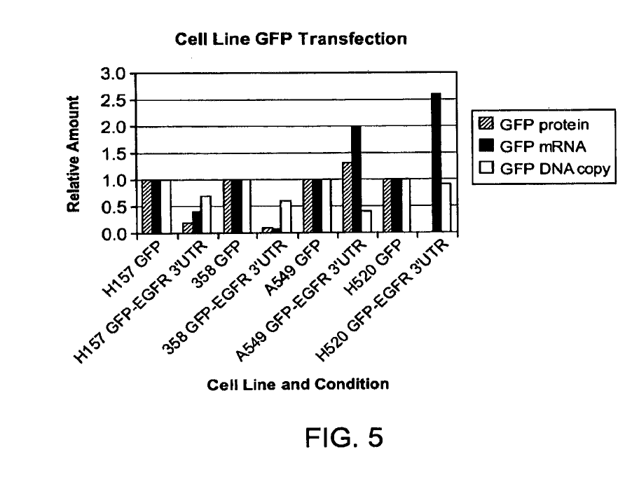

To show potential binding of miR-128 at EGFR-3′UTR, several cell lines were transfected with GFP and GPF-EGFR-3′UTR constructs. MiR-128b is predicted to bind at two loci in the EGFR-3′UTR. With binding occurring in these loci in the GFP-EGFR-3′UTR construct, degradation of GFP protein or message would be measurable. In three of four cell lines tested, GFP protein decreased by at least 83% (range 83-100%) and GFP mRNA decreased by 60-94% (FIGS. 4 and 5). GFP DNA copy number was determined to measure plasmid transfection and in the three cell lines demonstrating change, there was relatively similar plasmid cDNA, indicating equivalent transfection between GFP and GFP-EGFR-3′UTR among those cell lines.

To determine whether copy number of miR-128a or miR-128b correlates with clinical response or survival in NSCLC patients treated with gefitinib, we performed quantitative PCR on DNA extracted from microdissected primary NSCLC tumors samples from Tokyo Medical University (miR-128b data shown in Table 4). The tumors included 52 lung adenocarcinomas, 9 squamous cell carcinomas, and 1 large cell carcinoma from 38 male and 24 female patients, all of whom had progressed to stage 4 lung cancer and went on to receive treatment with EGFR-TKI gefitinib. DNA deletions specific to the miR-128b locus were frequent (56%, n=59). High EGFR IHC intensity was associated with disease presentation at a later stage (p=0.011).

Adenocarcinoma histology and women were significantly associated with improved response/disease control to gefitinib treatment (p=0.033 and p=0.001, respectively), findings supported by others (Dziadziuszko et al. 2006 Clin. Cancer Res. 12: 4409s; Miller et al. 2004 J. Clin. Oncol. 21: 3798; Kaneda et al. 2004 Lung Cancer 46:

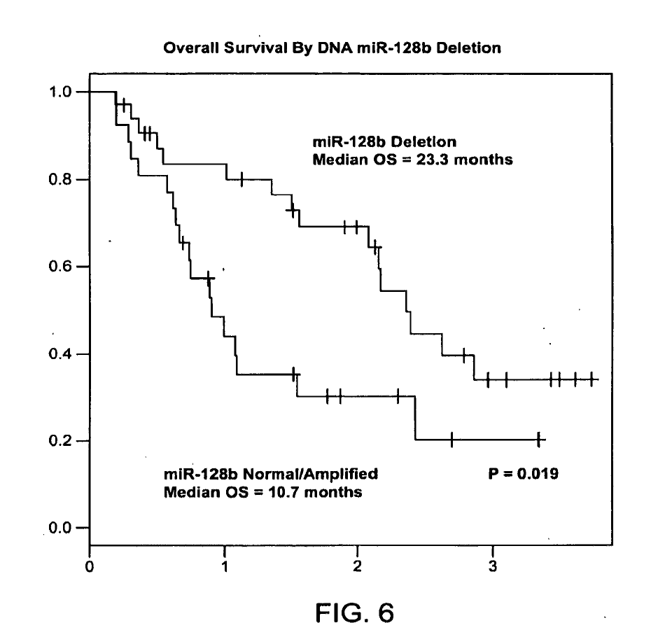

247). Deletion of mir-128b was also significantly associated with improved response/disease control to gefitinib treatment (p=0.026). Improved survival after initiation of gefitinib therapy was observed with adenocarcinoma histology (p=0.01), ≦3 lines of therapy (p=009), and miR-128b DNA deletion (p=0.019). Similarly, but in a smaller sample size (n=32), deletion of miR-128b DNA in combination with deletion of miR-128a DNA correlates with response and survival in these same patients (p=0.048). This data indicates that deletion of the miR-128a DNA also plays a role. However, in the smaller sample size, miR-128b DNA deletion alone highly correlated with survival and response to gefitinib treatment (p=0.08). EGFR IHC intensity had no correlation with survival (p=0.75) and patients aged 70 and older had similar benefit compared to their younger counterparts (p=0.22). These findings correlate with other reports that adenocarcinoma and line of treatment (Puijenbroek et al. 2007 Eur. Respir. J. 29: 128), but not EGFR IHC (Parra et al. 2004 Br. J. Cancer 91: 208; Bailey et al. 2003 Proc. Am. Assoc. Cancer Res. 44: 170A) and age (Kaneda et al. 2004 Lung Cancer 46: 247) are significantly associated with improved outcome. As patients are subjected to increasing lines of therapy, the likelihood of response and benefit diminish, and it is therefore not surprising that patients that received gefitinib ≦3rd-line therapy had improved survival. The significant correlation of deletion of miR-128b by DNA copy with improved survival, 23.3 months vs. 10.7 months, respectively (FIG. 6) has some biologic relevance. Others have shown a high concordance (73.1%) between miR DNA copy loss and mature miR expression (Zhang et al. 2006 Proc. Natl. Acad. Sci. U.S.A 103: 9136).

Based on the in vitro work, loss of miR-128b can be associated with increased EGFR protein or message expression. Gefitinib was developed as an EGFR-TKI and clearly has effects on miR-128b and EGFR message in a subset of NSCLC lines. Without being held to theory, it may be hypothesized that clinical response to gefitinib is improved in patients with tumors lacking miR-128b as more EGFR message and protein should be available for the EGFR-TKI to target.

Chromosome 3p loss is a common and early event in lung carcinogenesis. EGFR dysregulation is a frequent finding and important in cell growth, proliferation, and other events in lung cancer. With knowledge of miR involvement in cancer (Calin et al. 2002 Proc. Natl. Acad. Sci. U.S.A. 99: 15524) and using bioinformatics resources, the inventors conceived of linking this genetic abnormality to a dysregulated event in lung cancer. The locus of mir-128b on 3p22.3 is highly conserved in several species (http://genome.ucsc.edu/cgi-bin/hgTracks?hgsid=90958812&db=hg18&position=chr3%3A35760972-35761055). The Ras gene has been implicated in lung cancer and loss of let-7 family has been proposed to regulate this gene (Johnson et al. 2005 Cell 120: 635). Let-7g resides on chromosome 3p21.2 (http://www.ensembl.org/Homo_sapiens/contigview?1=3:52275334-52279417). Expression levels of let-7g were an average 30% less than normal in adjacent tissue in seven of eight samples (Johnson et. al. 2005 Cell 120: 635).

From the cell line and clinical specimen analyses, loss in 3p22.3 is frequent. The in vitro work demonstrates the impact of miR-128b deletion on EGFR expression. Transfection with miR-128b mimic and inhibitor alter EGFR levels, as would be expected with microRNA regulation in a subset of lines. Treatment of cell lines with gefitinib at doses above the IC₅₀, were positively correlated with EGFR and miR-128b levels, suggesting that alteration of miR-128b level is associated with cellular stress induced by an EGFR-TKI. MiR level change has been demonstrated in cytotoxic therapy (Meng et al. 2006 Gastroenterology 130: 211) and with additional signaling effects downstream of EGFR with miR-128b mimic or inhibitor transfection, may explain why synergistic growth inhibition was not observed with the co-transfection of gefitinib and mir-128b mimic or inhibitor. Loss of GFP expression in cell lines transfected with GFP-EGFR-3′UTR suggests that in a subset of lines, miR binding is occurring in the EGFR-3′UTR. Deletion of miR-128b copy number and its significant correlation in patient response and survival with gefitinib treatment applies in vitro findings of miR-128b regulation of EGFR and helps elucidate a biologic explanation for the impact of chromosomal loss in one area of the genome on dysregulated or amplified regions on other chromosomes.

TABLE 4
Patient Population

| | Gender/ | Treatment | | Surgical | Smok. | Relative DNA | EGFR | Best | Follow- | |
|---|---|---|---|---|---|---|---|---|---|---|
| Pt | Start Age | Line | Histology | Stage | Hist. | miR-128b copies | IHC Intensity | Resp. | up (days) | Stat |
| 1 | F/66 | 3 | Adeno | 3B | Never | 0.48 | 210 | SD | 1368 | 0 |
| 2 | F/67 | 5 | Squamous | 3A | Never | 1.94 | 290 | SD | 322 | 1 |
| 3 | M/70 | 2 | Adeno | 1B | Ever | 0.02 | 330 | SD | 369 | 1 |
| 4 | F/68 | 2 | Adeno | 3A | Never | 0.10 | N/A | SD | 1047 | 1 |
| 5 | M/75 | 3 | Large | 1B | Ever | 0.11 | 320 | PD | 189 | 1 |
| 6 | M/63 | 1 | Adeno | 2B | Ever | 0.21 | 220 | SD | 549 | 1 |
| 7 | F/72 | 2 | Adeno | 3A | Ever | 0.30 | 360 | SD | 91 | 0 |
| 8 | M/73 | 4 | Adeno | 1A | Ever | 0.12 | 140 | PR | 862 | 1 |
| 9 | M/61 | 4 | Adeno | 3B | Ever | 0.33 | N/A | SD | 181 | 1 |
| 10 | M/40 | 1 | Adeno | 3A | Never | 0.07 | 320 | SD | 761 | 1 |
| 11 | F/52 | 2 | Squamous | 3B | Never | 0.05 | 300 | SD | 957 | 1 |
| 12 | F/60 | 2 | Adeno | 3B | Never | 0.32 | 160 | SD | 569 | 1 |
| 13 | M/59 | 2 | Adeno | 1B | Ever | 0.11 | 210 | SD | 1084 | 0 |
| 14 | F/56 | 3 | Adeno | 3A | Never | 0.12 | 320 | SD | 131 | 1 |
| 15 | M/48 | 2 | Adeno | 1B | Ever | 0.13 | 110 | SD | 1020 | 0 |
| 16 | M/51 | 2 | Adeno | 1B | Ever | 2.65 | 220 | SD | 329 | 1 |
| 17 | M/53 | 2 | Adeno | 1A | Never | 5.33 | 280 | SD | 395 | 1 |
| 18 | F/64 | 2 | Adeno | 1B | Never | 0.45 | 230 | PR | 779 | 0 |
| 19 | M/70 | 3 | Squamous | 3A | Ever | 1.87 | 400 | PD | 104 | 1 |
| 20 | M/71 | 2 | Squamous | 3B | Ever | 31.84 | N/A | PD | 132 | 1 |
| 21 | M/53 | 2 | Adeno | 1A | Ever | 0.12 | 230 | SD | 694 | 0 |
| 22 | F/59 | 3 | Adeno | 1B | Never | 0.52 | 270 | SD | 686 | 0 |
| 23 | M/70 | 2 | Adeno | 1A | Never | 0.09 | N/A | PD | 65 | 1 |
| 24 | F/73 | 5 | Adeno | 3A | Ever | 0.69 | 370 | PR | 320 | 0 |
| 25 | F/58 | 3 | Adeno | 3B | Never | 1.75 | 320 | PR | 251 | 0 |
| 26 | F/76 | 2 | Squamous | 3A | Ever | 10.35 | 210 | PD | 110 | 1 |
| 27 | F/74 | 3 | Adeno | 4 | Never | 0.76 | 390 | CR | 985 | 0 |
| 28 | F/71 | 2 | Adeno | 3A | Never | 1.03 | N/A | SD | 886 | 1 |
| 29 | M/56 | 2 | Adeno | 3A | Never | 6.83 | N/A | SD | 562 | 1 |
| 30 | M/69 | 5 | Adeno | 3B | Never | 3.15 | 220 | SD | 224 | 1 |
| 31 | M/67 | 3 | Adeno | 1A | Ever | 2.07 | 220 | PD | 208 | 1 |
| 32 | M/71 | 2 | Squamous | 2B | Ever | 1.03 | N/A | SD | 271 | 1 |
| 33 | F/59 | 3 | Adeno | 3A | Ever | 1.80 | 300 | SD | 362 | 1 |
| 34 | M/58 | 2 | Adeno | 1A | Never | 0.13 | 160 | CR | 1256 | 0 |
| 35 | M/33 | 3 | Adeno | 3B | Never | 0.46 | 290 | PR | 789 | 1 |
| 36 | M/56 | 4 | Adeno | 1A | Ever | 1.61 | N/A | N/A | 231 | 1 |
| 37 | M/57 | 1 | Adeno | 3A | Ever | 0.30 | 400 | SD | 875 | 1 |
| 38 | M/63 | 5 | Adeno | 1B | Ever | 4.08 | 100 | PD | 71 | 1 |
| 39 | F/69 | 3 | Adeno | 1A | Never | 13.53 | N/A | SD | 399 | 1 |
| 40 | M/45 | 2 | Adeno | 1A | Ever | 1.54 | N/A | SD | 553 | 0 |
| 41 | M/43 | 1 | Adeno | 3A | Ever | 1.17 | 180 | PD | 268 | 1 |
| 42 | M/65 | 4 | Adeno | 4 | Never | 3.48 | N/A | SD | 242 | 1 |
| 43 | M/62 | 2 | Adeno | 3A | Ever | 2.27 | 360 | PR | 648 | 0 |
| 44 | F/72 | 1 | Adeno | 1B | Never | 1.11 | 350 | CR | 838 | 0 |
| 45 | M/65 | 2 | Adeno | 3A | Ever | 9.58 | 380 | PD | 71 | 1 |
| 46 | F/52 | 2 | Adeno | 1B | Ever | 0.02 | N/A | SD | 146 | 0 |
| 47 | F/60 | 2 | Adeno | 1A | Never | 0.03 | 400 | PR | 1133 | 0 |
| 48 | F/64 | 2 | Adeno | 3A | Ever | 0.01 | 300 | SD | 197 | 1 |
| 49 | F/73 | 2 | Adeno | 2B | Never | 0.03 | 250 | CR | 491 | 1 |
| 50 | M/64 | 2 | Adeno | 3A | Ever | 0.06 | 400 | PR | 1278 | 0 |
| 51 | M/70 | 3 | Squamous | 2B | Never | 0.03 | N/A | SD | 112 | 1 |
| 52 | M/49 | 2 | Adeno | 3A | Ever | 0.01 | 400 | SD | 553 | 0 |
| 53 | M/65 | 2 | Adeno | 1B | Ever | 0.04 | 130 | PD | 161 | 0 |
| 54 | F/69 | 3 | Adeno | 3B | Never | 0.01 | N/A | SD | 1321 | 0 |
| 55 | F/27 | 3 | Adeno | 3A | Ever | 0.04 | 360 | SD | 410 | 0 |
| 56 | M/57 | 2 | Squamous | 1B | Ever | 0.00 | N/A | N/A | 797 | 1 |
| 57 | M/61 | 3 | Squamous | 2B | Ever | 0.00 | 400 | PR | 698 | 0 |
| 58 | F/42 | 1 | Adeno | 1A | Never | 0.15 | 320 | PR | 727 | 0 |
| 59 | M/54 | 3 | Adeno | 3B | Ever | 7.78 | N/A | PD | 1222 | 0 |
| 60 | M/65 | 3 | Adeno | 1A | Ever | N/A | 220 | SD | 907 | 0 |
| 61 | M/59 | 2 | Adeno | 4 | Ever | N/A | N/A | SD | 898 | 0 |
| 62 | M/76 | 2 | Adeno | 4 | Never | N/A | 320 | SD | 643 | 0 |

Key:
Adeno = adenocarcinoma
Squamous = squamous cell carcinoma
N/A = not available
Best Resp. = best response
PD = progressive disease
SD = stable disease
PR = partial response
CR = complete response
Stat = status
Alive = 0
Dead = 1
Pt = patient
Smok. Hist. = smoking history

### Sequences

Table 5 is a summary of sequences mentioned throughout the claims and specification along with their respective sequence identification numbers.

TABLE 5
Sequences and Sequence Identifiers

| SEQ | | |
|---|---|---|
| ID NO | Description | Sequence 5′-3′ |
| 1 | Forward primer 3p22 | AGGTACAAGAAGGTGAAGCA |
| | encompassing miR-128b | |
| 2 | Reverse primer 3p22 | GATGTCTGTGATTGGTGCTA |
| | encompassing miR-128b | |
| 3 | Forward primer EGFR | ATTAGCTCTTAGACCCACAGACTGG |
| | 3′UTR binding site 1 | |
| 4 | Reverse primer EGFR | TTCTTGCTGGATGCGTTTCTGTAAAT |
| | 3′UTR binding site 1 | |
| 5 | Forward primer EGFR | TACCCTGAGTTCATCCAGGCC |
| | 3′UTR binding site 2 | |
| 6 | Reverse primer EGFR | AGTGGAAGCCTTGAAGCAGAAC |
| | 3′UTR binding site 2 | |
| 7 | Forward miR-128b primer | GCCGATACACTGTACGAGAGTGA |
| 8 | Reverse miR-128b primer | GGAGTGTGACACAGTAGGGAAAGA |
| 9 | Forward CFTR primer | CGCGATTTATCTAGGCATAGGC |
| 10 | Reverse CFTR primer | TGTGATGAAGGCCAAAAATGG |
| 11 | miR-128b probe | 6FAM-TAGCAGGTCTCACAGTGAACCGGT- |
| | | TAMRA |
| 12 | CFTR probe | VIC-TGCCTTCTCTTTATTGTGAGGACACTG |
| | | CTCC-TAMRA |
| 13 | GFP forward primer | CGACAAGCAGAACAACGGCATCAA |
| 14 | GFP reverse primer | AACTCCAGCAGGACCATGTGA |
| 15 | Beta-actin forward | ATCCACGAAACTACCTTCAACTC |
| 16 | Beta-actin reverse | GAGGAGCAATGATCTTC |
| 17 | EGFR 3′UTR by 19-554 | (See Fig. 1) |
| 18 | miR-128a DNA | TGAGCTGTTGGATTCGGGGCCGTAGCACT |
| | | GTCTGAGAGGTTTACATTTCTCACAGTGA |
| | | ACCGGTCTCTTTTTCAGCTGCTTC |
| 19 | miR-128a RNA | UGAGCUGUUGGAUUCGGGGCCGUAGCA |
| | | CUGUCUGAGAGGUUUACAUUUCUCACA |
| | | GUGAACCGGUCUCUUUUUCAGCUGCUUC |
| 20 | miR-128b DNA | TGTGCAGTGGGAAGGGGGGCCGATACA |
| | | CTGTACGAGAGTGAGTAGCAGGTCTCA |
| | | CAGTGAACCGGTCTCTTTCCCTACTGTGTC |
| 21 | miR-128b RNA | UGUGCAGUGGGAAGGGGGGCCGAUACA |
| | | CUGUACGAGAGUGAGUAGCAGGUCUCA |
| | | CAGUGAACCGGUCUCUUUCCCUACUGU |
| | | GUC |
| 22 | Mature miR-128a RNA | UCACAGUGAACCGGUCUCUUUU |
| 23 | Mature miR-128b RNA | UCACAGUGAACCGGUCUCUUUC |
| 24 | Forward miR-128b primer | GGGCCGTAGCACTGTCTGA |
| 25 | Reverse miR-128b primer | CCAGGAAGCAGCTGAAAAAGA |
| 26 | miR-128a probe | TTTACATTTCTCACAGTGAACCGGT |

TABLE 6
Cox proportional hazards; complete and modified models

| Variable³ | Hazard ratio | 95% CI | P value |
|---|---|---|---|
| Complete model | | | |
| Sex | | | |
| Female/male | 0.35 | 0.13-0.96 | 0.04 |
| Age (years) | | | |
| <70/≧70 | 0.60 | 0.23-1.55 | 0.29 |
| Histology | | | |
| Adenocarcinoma/squamous | 0.32 | 0.13-0.84 | 0.02 |
| Smoking status | | | |
| Former, current/never | 0.78 | 0.32-1.90 | 0.58 |
| Stage | | | |
| I-II/III-IV | 0.71 | 0.31-1.65 | 0.43 |
| Lines of treatment | | | |
| ≦3/>3 | 0.42 | 0.16-1.12 | 0.08 |
| microRNA-128b | | | |
| LOH/no LOH | 0.49 | 0.20-1.17 | 0.11 |
| EGFR mutation/deletion status | | | |
| Exon 19 deletion/no exon 19 | 0.77 | 0.30-2.01 | 0.60 |
| deletion | | | |
| Exon 21 point mutation/no | 1.18 | 0.36-3.89 | 0.78 |
| exon 21 point mutation | | | |
| Modified model | | | |
| Histology | | | |
| Adenocarcinoma/squamous | 0.38 | 0.17-0.86 | 0.02 |
| Lines of treatment | | | |
| ≦3/>3 | 0.36 | 0.14-0.89 | 0.03 |
| microRNA-128b | | | |
| LOH/no LOH | 0.45 | 0.22-0.93 | 0.03 |

³All variables codes as true/false in order listed.

CI, confidence interval; EGFR, epidermal growth factor receptor; LOH, loss of heterozygosity

All references cited above are incorporated herein by reference in their entirety.

The words “comprise”, “comprises”, and “comprising” are to be interpreted inclusively rather than exclusively.

What is claimed is:

1. A method for identifying a cancer patient responsive to treatment with an EGFR tyrosine kinase inhibitor, the method comprising detecting a genomic loss of miR-128b in a cancer biopsy obtained from said cancer patient, wherein said genomic loss of miR-128b indicates said cancer patient is responsive to treatment with an EGFR tyrosine kinase inhibitor.

2. The method of claim 1, wherein the genomic loss is assessed by measuring the number of copies of miR-128b in DNA extracted from the cancer biopsy by performing quantitative PCR on miR-128b DNA extracted from the cancer biopsy.

3. The method of claim 2, wherein the performing quantitative PCR comprises use of a forward primer having at least 50% sequence identity to SEQ ID NO: 7 and a reverse primer having at least 50% sequence identity to SEQ ID NO: 8.

4. The method of claim 3, wherein the forward primer comprises nucleotides extending up to 1, 2, 3, 4, or 5 nucleotides upstream or downstream of SEQ ID NO: 7.

5. The method of claim 3, wherein the reverse primer comprises nucleotides extending up to 1, 2, 3, 4, or 5 nucleotides upstream or downstream of SEQ ID NO: 8.

6. The method of claim 2, wherein the measuring the number of copies of miR-128b in DNA extracted from the biopsy comprises using a probe having at least 50% sequence identity to SEQ ID NO: 11.

7. The method of claim 6, wherein the probe comprises nucleotides extending up to 1, 2, 3, 4, or 5 nucleotides upstream or downstream of SEQ ID NO: 11.

8. The method of claim 1, wherein the cancer is a lung cancer.

9. The method of claim 8, wherein the cancer is selected from the group consisting of squamous cell carcinoma, adenocarcinoma, large cell carcinoma, and combinations thereof.

10. The method of claim 8, wherein the cancer is non-small-cell lung cancer.

11. A method for identifying a cancer patient responsive to treatment with an EGFR tyrosine kinase inhibitor, the method comprising measuring miR-128b or miR-128a level in a biopsy obtained from the patient and comparing that level to an unaffected gene in the same tissue sample, wherein a patient responsive to treatment with an EGFR tyrosine kinase inhibitor has a cancer expressing a lower level of miR-128b or miR-128a relative to the unaffected gene.

12. The method of claim 11, wherein the unaffected gene is selected from the group consisting of CFTR, beta actin, and tubulin.

13. A method for treating cancer in a patient in need thereof, the method comprising:
(a) measuring the level of miR-128b or miR-128a in a biopsy obtained from the patient; and
(b) administering to the patient an EGFR tyrosine kinase inhibitor.

14. The method of claim 10, wherein the level of miR-128b is measured and is underexpressed relative to an unaffected gene.

15. The method of claim 10, wherein the level of miR-128b is measured and is overexpressed relative to an unaffected gene, and wherein the patient is further administered a miR-128b inhibitor.

16. A method for treating cancer in a patient in need thereof, the method comprising:
(a) measuring a ratio of the number of copies of miR-128b to the number of copies of an unaffected gene in DNA extracted from a biopsy obtained from the patient; and
(b) administering to the patient an EGFR tyrosine kinase inhibitor.

17. The method of claim 16, wherein the ratio is less than 0.5.

18. The method of claim 16, wherein the ratio is 0.5 or greater, and wherein the patient is further administered a miR-128b inhibitor.

19. A method for treating cancer, the method comprising administering to a cancer patient an EGFR tyrosine kinase inhibitor and a miR-128b inhibitor.

20. The method of claim 19, wherein the EGFR tyrosine kinase inhibitor is selected from the group consisting of gefitinib, erlotinib, and any other EGFR-tyrosine kinase inhibitor.

21. The method of claim 19, wherein the miR-128b inhibitor is selected from the group consisting of antisense molecules, aptamers, siRNAs, and oligonucleotides.

22. The method of claim 19, wherein the EGFR tyrosine kinase inhibitor and miR-128b inhibitor are administered together.

23. The method of claim 19, wherein the miR-128b inhibitor is administered prior to administration of the EGFR tyrosine kinase inhibitor.

24. The method of claim 19, wherein the EGFR tyrosine kinase inhibitor and the miR-128b inhibitor are administered over the course of several hours to several months.

25. A method for treating cancer, the method comprising administering to a cancer patient a composition comprising a miR-128b

26. A method for treating cancer, the method comprising administering to a cancer patient a composition comprising a miR-128a mimic.

27. A method for treating cancer, the method comprising administering to a cancer patient a composition comprising a miR-128a inhibitor and an EGFR tyrosine kinase inhibitor.

28. A composition used to treat cancer in a patient, the composition comprising an EGFR tyrosine kinase inhibitor and a miR-128b inhibitor, wherein the cancer is characterized as having a ratio of 0.5 or greater of miR-128b DNA to an unaffected gene at the cellular level.

29. The composition of claim 28, wherein the EGFR tyrosine kinase inhibitor is selected from the group consisting of gefitinib, erlotinib, and any other EGFR-tyrosine kinase inhibitor.

30. The composition of claim 28, wherein the miR-128b inhibitor is selected from the group consisting of monoclonal antibodies, polyclonal antibodies, antisense molecules, aptamers, siRNAs, and oligonucleotides.

31. The composition of claim 30, wherein the miR-128b inhibitor is an oligonucleotide that binds to miR-128b.

32. The composition of claim 28, wherein the miR-128b inhibitor physically interacts with miR-128b.

33. The composition of claim 28, wherein the miR-128b inhibitor acts to inhibit miR-128b by preventing it from binding to a miR-128b binding site on a 3′ untranslated region of EGFR mRNA.

34. The composition of claim 28, wherein the EGFR tyrosine kinase inhibitor is gefitinib and the miR-128b inhibitor is an oligonucleotide that binds to miR-128b.

35. A method for identifying a cancer therapeutic, the method comprising screening for compounds that target a miR-128b binding site or miR-128a binding site on a 3′ untranslated region of EGFR mRNA.

36. A method for identifying a cancer therapeutic, the method comprising screening for compounds that inhibit miR-128b, wherein the therapeutic is used in combination with an EGFR tyrosine kinase inhibitor to treat a cancer that overexpresses miR-128b.

37. A method for identifying a patient or patient population predisposed to cancer, the method comprising measuring the level of miR-128b, number of miR-128b DNA copies, or both in a biopsy obtained from the patient.


SEQUENCE LISTING (26 sequences)

SEQ ID NO: 1 - DNA, 20 bp, Artificial
  Synthetic forward primer 3p22 encompassing miR-128b
aggtacaagaaggtgaagca

SEQ ID NO: 2 - DNA, 20 bp, Artificial
  Synthetic reverse primer 3p22 encompassing miR-128b
gatgtctgtgattggtgcta

SEQ ID NO: 3 - DNA, 25 bp, Artificial
  Synthetic forward primer EGFR 3′UTR binding site 1
attagctcttagacccacagactgg

SEQ ID NO: 4 - DNA, 26 bp, Artificial
  Synthetic reverse primer EGFR 3′UTR binding site 1
ttcttgctggatgcgtttctgtaaat

SEQ ID NO: 5 - DNA, 21 bp, Artificial
  Synthetic forward primer EGFR 3′UTR binding site 2
taccctgagttcatccaggcc

SEQ ID NO: 6 - DNA, 22 bp, Artificial
  Synthetic reverse primer EGFR 3′UTR binding site 2
agtggaagccttgaagcagaac

SEQ ID NO: 7 - DNA, 23 bp, Artificial
  Synthetic forward miR-128b primer
gccgatacactgtacgagagtga

SEQ ID NO: 8 - DNA, 24 bp, Artificial
  Synthetic reverse miR-128b primer
ggagtgtgacacagtagggaaaga

SEQ ID NO: 9 - DNA, 22 bp, Artificial
  Synthetic forward CFTR primer
cgcgatttatctaggcataggc

SEQ ID NO: 10 - DNA, 21 bp, Artificial
  Synthetic reverse CFTR primer
tgtgatgaaggccaaaaatgg

SEQ ID NO: 11 - DNA, 24 bp, Artificial
  Synthetic miR-128b probe
tagcaggtctcacagtgaaccggt

SEQ ID NO: 12 - DNA, 31 bp, Artificial
  Synthetic CFTR probe
tgccttctctttattgtgaggacactgctcc

SEQ ID NO: 13 - DNA, 24 bp, Artificial
  Synthetic GFP forward primer
cgacaagcagaacaacggcatcaa

SEQ ID NO: 14 - DNA, 21 bp, Artificial
  Synthetic GFP reverse primer
aactccagcaggaccatgtga

SEQ ID NO: 15 - DNA, 23 bp, Artificial
  Synthetic Beta-actin forward primer
atccacgaaactaccttcaactc

SEQ ID NO: 16 - DNA, 17 bp, Artificial
  Synthetic Beta-actin reverse primer
gaggagcaatgatcttc

SEQ ID NO: 17 - DNA, 475 bp, Artificial
  Synthetic EGFR 3′UTR bp 19-554
gccctaaaaatccagactctttcgatacccaggaccaagccacagcaggtcctccatcccaacagccatgcccgcattagctcttagacccacagactggttttgcaacgtttacaccgactacccaggaagtacttccacctcgggcacattttgggaagttgcattcctttgtcttcaaactgtgaagcatttacagaaacgcatccagcaagaatattgtccctttgagcagaaatttatctttcaaagaggtatattgaaaaaaaaaaaaagtatatgtgaggatttttattgacaaggaagaaccttgctggtaccacttgctaccctgagttcatccaggcccaactctgagcaaggagcacaagccacaagtcttccagaggatgcttgattccagtggttctgcttcaaggcttccactgcaaaacactaaagatccaagaaggccttcatggccccagcaggccgg

SEQ ID NO: 18 - DNA, 82 bp, Artificial
  Synthetic miR-128a DNA
tgagctgttggattcggggccgtagcactgtctgagaggtttacatttctcacagtgaaccggtctctttttcagctgcttc

SEQ ID NO: 19 - RNA, 82 nt, Artificial
  Synthetic miR-128a RNA
ugagcuguuggauucggggccguagcacugucugagagguuuacauuucucacagugaaccggucucuuuuucagcugcuuc

SEQ ID NO: 20 - DNA, 84 bp, Artificial
  Synthetic miR-128b DNA
tgtgcagtgggaaggggggccgatacactgtacgagagtgagtagcaggtctcacagtgaaccggtctctttccctactgtgtc

SEQ ID NO: 21 - RNA, 84 nt, Artificial
  Synthetic miR-128b RNA
ugugcagugggaaggggggccgauacacuguacgagagugaguagcaggucucacagugaaccggucucuuucccuacuguguc

SEQ ID NO: 22 - RNA, 22 nt, Artificial
  Synthetic mature miR-128a RNA
ucacagugaaccggucucuuuu

SEQ ID NO: 23 - RNA, 22 nt, Artificial
  Synthetic Mature miR-128b RNA
ucacagugaaccggucucuuuc

SEQ ID NO: 24 - DNA, 19 bp, Artificial
  Synthetic forward miR-128b primer
gggccgtagcactgtctga

SEQ ID NO: 25 - DNA, 21 bp, Artificial
  Synthetic reverse miR-128b primer
ccaggaagcagctgaaaaaga

SEQ ID NO: 26 - DNA, 25 bp, Artificial
  Synthetic miR-128a probe
tttacatttctcacagtgaaccggt